(19) United States
(12) Patent Application Publication
(10) Pub. No.: US 2008/0220031 A1
(43) Pub. Date: Sep. 11, 2008

(54) Dermocosmetic Preparations

(75) Inventors: Thomas Wunsch, Speyer (DE); Sylke Haremza, Neckargemund (DE); Axel Jentzsch, Ludwigshafen (DE); Gerhard Wagenblast, Wachenheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/996,424

(22) PCT Filed: Jul. 14, 2006
(86) PCT No.: PCT/EP2006/064264

§ 371 Date: Jan. 22, 2008

(30) Foreign Application Priority Data
Jul. 25, 2005 (EP) 0510685.2

(51) Int. Cl. A61K 8/49; A61Q 5/00; A61Q 19/00; A61K 8/06

(52) U.S. Cl. 424/401; 546/208; 424/59; 424/70.9

(57) ABSTRACT

The present invention relates to dermocosmetic compositions comprising a compound comprising the structural element of the formula I

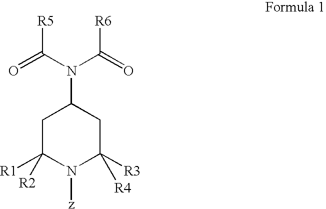

where the radical Z has the following meaning: H, C₁-C₂₂alkyl group, C₆-C₁₀-aryl group, C₁-C₂₂-alkoxy group or a C₆-C₁₀-O-aryl group substituted by a C₁-C₂₂-alkyl or C-C₂₂-alkoxy group,

and the radicals R¹ to R⁶, independently of one another, have the following meaning: H, C₁-C₂₂-alkyl group, C₆-C₁₀-aryl group, O, OH, C₁-C₂₂-alkoxy group or a C₆-C₁₀-O-aryl group substituted by a C₁-C₂₂-alkyl or C₁-C₂₂-alkoxy group,

it being possible for R⁵ and R⁶ to be bridged in such a way that a five- to eight-membered ring is formed and, in the case of a five-membered ring, this ring may be part of an oligomer as a result of covalent bonds at positions 3 and 4.

The invention furthermore relates to the use of said compounds in dermocosmetic formulations and the use of the dermocosmetics according to the invention for reducing skin or hair damage caused by free radicals. The invention furthermore relates to the use of the compounds according to the invention for increasing the stability of dermocosmetic formulations.

The present invention relates to dermocosmetic compositions comprising a compound comprising the structural element of the formula 1. The invention furthermore relates to the use of these compounds comprising the structural element of the general formula 1 in dermocosmetic formulations and the use of the dermocosmetics according to the invention for reducing or avoiding skin or hair damage caused by free radicals. The invention furthermore relates to the use of the compounds according to the invention for increasing the stability of dermocosmetic formulations or for stabilizing the oxidation-sensitive ingredients present in said formulations. Further embodiments of the present invention are described in the claims, the description and the examples. It is clear that the abovementioned features of the subject matter according to the invention and those features which are still to be explained below can be used not only in the combination stated in each case but also in other combinations without departing from the scope of the invention.

Skin aging is now understood to be the result of the interplay of chronological (intrinsic) skin aging and skin aging due to exogenous factors (extrinsic). It is assumed that intrinsic skin aging is the result of genetic processes.

Extrinsic skin aging is influenced by exogenous influences, in particular WV radiation (photoaging). The formation of free carbon radicals or reactive oxygen species (ROS), such as, for example, peroxide radicals, is regarded as a substantial mechanism which leads to extrinsic skin aging. These extremely reactive substances lead to oxidation of a very wide range of cell constituents, such as, for example, the DNA, proteins and membrane lipids. In the skin area, free radicals damage the collagen and the elastin and adversely affect the fatty substances. The skin thus loses its tone and its elasticity, and the age spot pigment lipofuscin forms. The free radicals form under the influence of numerous factors: environmental pollution, excessive action of sunlight, smoking and alcohol and fat consumption.

The exposure of the skin to sunlight is responsible for the premature aging of skin areas exposed to light. Human skin is highly sensitive in the short-wave radiation range (WV-B) having wavelengths of 290-320 nm. The WV-A range (320-400 nm) is predominantly responsible for chronic UV damage. Furthermore, the following clinical pictures are adversely affected by exposure to sunlight: Lupus erythematodes, solar urticaria, Hydroa vacciniformis, light-aggravated atopic dermatitis, Xeroderma pigmentosum, vitiligo, chronic actinic dermatitis, Herpes simplex infections and so-called Mallorca acne (triggered in appropriately disposed persons by peroxides with simultaneous exposure to UV radiation (phototoxic chemotoxic skin reaction)). Furthermore, the expression of metalloproteinases is induced by UVA radiation. This leads to the reduction of collagens and elastic fibers. Clinically, extrinsic skin aging typically occurs in the skin areas which are exposed to environmental influences. Here, they are superposed on the symptoms of intrinsic skin aging. Thus, the protection of the skin from the consequences of exposure to intense sunlight is of major importance for human health.

Furthermore, said harmful influences result in damage to the cells of the skin itself. As a result of this, for example, the regenerability of the skin is reduced. The results of aging are thinning of the skin, weaker intermeshing of epidermis and dermis and a reduction in the number of cells and of supplying blood vessels.

The same factors also act on hair, where damage can also occur. The hairs become brittle, less elastic and dull. The surface structure of the hairs is damaged.

In order to counteract the stress described above, the hair has a multiplicity of its own protection mechanisms. However, these protection mechanisms are not sufficient for completely preventing oxidative processes and the aging processes or damage to the skin associated therewith (cf. also “Skin diseases associated with oxidative injury” in “oxidative stress in dermatology”, page 323 et seq., Marcel Decker Inc., New York, Basle, Hong Kong, editors: Jürgen Fuchs, Frankfurt and Lester Packer, Berkeley/Calif.).

In particular, the importance of lipid peroxidation for aging is generally recognized. The toxic effect of lipid hydroperoxides and the decomposition products thereof was also described, inter alia, by A. Sevanian and P. Hochstein (Mechanisms and Consequences of Lipid Peroxidation in Biological Systems, Annual Review of Nutrition, Vol. 5: 365-390 (July 1985)).

The importance of free radicals in association with skin aging has led in recent years to an intensive search for active substances which eliminate the harmful effects of free radicals and thus protect the tissue from oxidative damage. Such compounds having an antioxidant effect are used in dermocosmetic or cosmetic formulations for protecting and caring for skin and hair. In addition, however, they can also be used for preventing the spoiling of oxidation-sensitive substances.

Cosmetic or dermocosmetic care products having properties which are intended to counteract the processes described or comparable processes or to reduce or cancel out the harmful consequences thereof are frequently distinguished by the following properties: free radical scavenging, antioxidant antiinflammatory or humectant. They prevent or reduce, inter alia, the activity of the matrix-degrading enzyme or regulate the resynthesis of collagen, elastin or proteoglycans.

In order to achieve the abovementioned properties, free radical scavengers are added to the dermocosmetic formulations (e.g. DE 19739349). Of particular importance thereby are the vitamins E (tocopherols) and C (ascorbic acid) which are added as free radical scavengers for oxygen radicals to said formulations. WO 2005/042828 describes the use of various hindered nitroxyls, hydroxylamines and hydroxylamine salts in combination with organic UV filters for stabilizing personal hygiene products. However, there are few known substances which can be used for effective deactivation of carbon radicals for protecting the skin and/or of oxidation-sensitive dermocosmetically active substances.

In addition, the actually achieved effect of deactivation of carbon radicals by the substances or enzymatic systems described has to date not achieved the effects hoped for.

Owing to the constantly increasing requirement for dermocosmetic active substances for preventing the abovementioned damage to skin and/or hair or reducing existing damage to skin and/or hair, in particular as a result of phototoxic chemotoxic reactions, it was the object of the present invention to identify active substances which (i) have as little irritation potential as possible for the skin, (ii) have a high free radical-deactivating effect and (iii) are also suitable for the preparation of cosmetic and/or dermocosmetic formulations or preparations. In particular, it was the object of the present invention to provide active substances and compositions comprising these active substances, which active substances and compositions have a free radical-decomposing effect and can be used for avoiding or reducing skin and/or hair damage caused by said free radicals. It was furthermore of particular interest to identify active substances which stabilize oxidation-sensitive dermocosmetically active substances and prolong the stability of such formulations. It was therefore also an object of the invention to provide dermocosmetic formulations which are suitable for the prophylaxis and treatment of light-sensitive skin, particularly of photodermatoses and polymorphic photodermatoses as described in the literature (e.g. Aa. Voelckel et al., Zentralblatt Haut-und Geschlechtskranlheiten (1989), 156, page 2). In particular, it was an object of the present invention to identify substances which are suitable for deactivating both carbon and peroxide radicals and therefore provide comprehensive protection from free radicals.

Surprisingly, it was found that compounds which comprise a structural element of the general formula I have the desired properties and that the objects set could be achieved by the use thereof in dermocosmetic formulations.

Sterically hindered amines or “hindered amine stabilizers” (HAS) are derivatives of 2,2,6,6-tetramethylpiperidine. HAS are now the most important sunscreen agents in the area of the polyolefins. HAS have been used in the chemical industry since the beginning of the 1990s for stabilizing polymers to oxidation damage (R. L. Gray. A Novel nonreactive HALS boosts polyolefin stability. Plastics engineering, 1991, 21). Through their action in decomposing peroxide radicals, HAS prevent the light-induced decomposition of plastics and plastics products. Thus, HAS stabilize plastics by scavenging free radicals formed as intermediates, a free radical scavenging cycle according to the following scheme being assumed as the action mechanism:

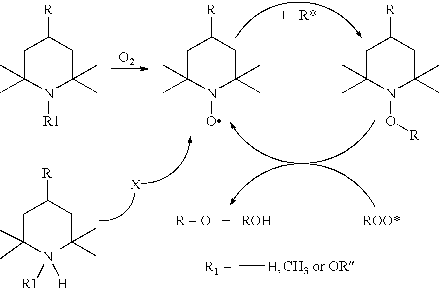

A skin-irritating effect is attributed to the HAS. Characteristically, HAS are basic molecules and are therefore present in the protonated form in the pH-neutral to slightly acidic pH range, as is customary for cosmetic or dermocosmetic formulations. Since the presence of a deprotonated form of the HAS is necessary for the free radical scavenging cycle described above to take place efficiently (cf. FIG. 1) and HAS are protonated and hence deactivated by acids, the effectiveness of HAS as free radical scavengers would thus be reduced in a pH-neutral to slightly acidic medium, with the result that use in dermocosmetic formulations is ruled out.

Furthermore, HAS, in particular oligomeric HAS, are as a rule not soluble (or soluble only to an insufficient degree) in cosmetic oils (cf. example 2).

Taking into account said properties of the HAS, it was surprising that compounds which comprise a structural element of the general formula 1 (i) have a high free radical-decomposing activity in a pH-neutral to slightly acidic medium, (ii) also have a solubility in cosmetic oils which is sufficient for the preparation of cosmetic or dermocosmetic formulations and (iii) have good skin tolerance.

### SUMMARY OF THE INVENTION

The present invention relates to dermocosmetics comprising at least one compound comprising the structural element of the general formula 1

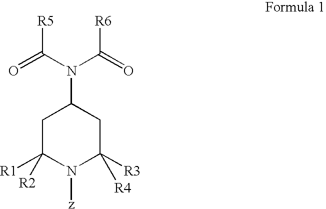

where the radical Z has the following meaning: H, C₁-C₂₂-alkyl group, C₆-C₁₀-aryl group, C₁-C₂₂-alkoxy group or a C₆-C₁₀—O-aryl group substituted by a C₁-C₂₂-alkyl or C₁-C₂₂-alkoxy group,

and the radicals R¹ to R⁶, independently of one another, have the following meaning: H, C₁-C₂₂-alkyl group, C₆-C₁₀-aryl group, O, OH, C₁-C₂₂-alkoxy group or a C₆-C₁₀—O-aryl group substituted by a C₁-C₂₂-alkyl or C₁-C₂₂-alkoxy group,

it being possible for R⁵ and R⁶ to be bridged in such a way that a five- to eight-membered ring is formed and, in the case of a five-membered ring, this ring may be part of an oligomer as a result of covalent bonds at positions 3 and 4.

A further embodiment of the invention relates to dermocosmetics which comprise an oligomer of the general formula 2

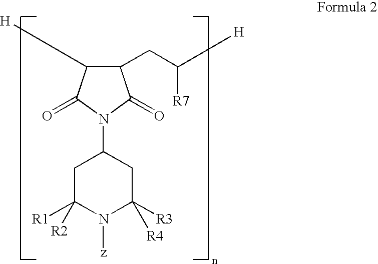

where the radical Z has the following meaning: H, C₁-C₂₂-alkyl group, C₆-C₁₀-aryl group, C₁-C₂₂-alkoxy group or a C₆-C₁₀—O-aryl group substituted by a C₁-C₂₂-alkyl or C₁-C₂₂-alkoxy group,

and the radicals R¹ to R⁶, independently of one another, have the following meaning: H, C₁-C₂₂-alkyl group, C₆-C₁₀-aryl group, O, OH, C₁-C₂₂-alkoxy group or a C₆-C₁₀—O-aryl group substituted by a C₁-C₂₂-alkyl or C₁-C₂₂-alkoxy group,

and R⁷ corresponds to a C₁- to C₃₀-alkyl group and n is an integer from 2 to 1000.

In a particularly preferred embodiment the dermocosmetics comprise at least one compound of the formula 3

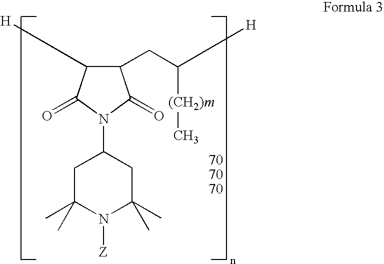

where Z may be an H or a C₁-C₂₂-alkyl group and n corresponds to an integer from 2 to 1000 and m corresponds to an integer from 10 to 30.

The invention furthermore relates to dermocosmetics comprising a compound of the general formula 4

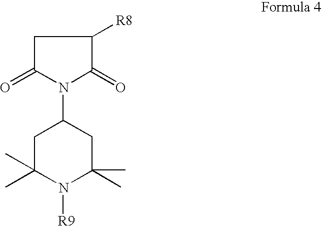

where the radicals, independently of one another, have the following meaning:

R⁸: C₁-C₂₅-alkyl group and R⁹: H or a C₁-C₂₂-alkyl group or a C₁-C₂₂-alkoxy group.

The present invention particularly preferably relates to dermocosmetics comprising a sterically hindered amine selected from the group consisting of 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and 3-octenyl-N-(1,2,2,6,6-pentamethyl-4-piperidinyl)succinimide.

In another preferred embodiment, the present invention also comprises cosmetic compositions for oral, dental and dental prosthesis care, these compositions comprising at least one compound according to the invention, according to the formulae 1 to 4.

In a preferred embodiment of the present invention, the dermocosmetics comprise a compound according to the invention in a concentration of from 0.001 to 30 percent by weight, based on the total weight of the composition.

The invention furthermore relates to dermocosmetics comprising a compound according to the invention of the general formulae 1 to 4, the nitrogen atom of the piperidine ring of said compound being present in deprotonated form at a pH suitable for dermocosmetics.

In a preferred embodiment, the dermocosmetics according to the invention comprise, in addition to the abovementioned compounds, one or more cosmetic or dermocosmetic active substances. These are preferably active substances selected from the group consisting of the natural or synthetic polymers, pigments, humectants, oils, waxes, enzymes, minerals, vitamins, sunscreen agents, dyes, fragrances, antioxidants and preservatives.

The dermocosmetics according to the invention are preferably skin protection compositions, skin care compositions, skin cleansing compositions, hair protection compositions, hair setting compositions, hair cleansing compositions or formulations for decorative cosmetics, which are preferably used in the form of ointments, creams, emulsions, suspensions, lotions, as milk, pastes, gels, foams or sprays.

The invention furthermore relates to the use of the compounds according to the invention according to the formulae 1 to 4 as an additive for the preparation of dermocosmetics. In a preferred embodiment, the abovementioned compounds according to the invention are used for stabilizing light- and/or oxidation-sensitive substances in dermocosmetics.

The compounds according to the invention according to the formulae 1 to 4 are preferably used for the preparation of skin protection compositions, skin care compositions, skin cleansing compositions, hair protection compositions, hair setting compositions, hair cleansing compositions or in formulations for decorative cosmetics.

A further preferred embodiment of the present invention relates to the use of the compounds according to the invention according to the formulae 1 to 4 in dermocosmetics for avoiding skin or hair damage caused by free radicals.

In a preferred embodiment of the present invention, compounds comprising a structural element of the general formula 1 to 4 are used, in which the nitrogen atom of the piperidine ring is present in deprotonated form at the pH suitable for cosmetic or dermocosmetic compositions.

In a preferred embodiment of the present invention, the compounds according to the invention according to the formulae 1 to 4 are used in a concentration of from 0.001 to 30 percent by weight, based on the total weight of the composition.

Definitions

“Decorative cosmetics” means cosmetic assistants which are used not primarily for care but for enhancement or improvement of the appearance. Relevant assistants of this type are known to the person skilled in the art and comprise, for example, kohl sticks, mascara, eyeshadows, tinted day creams, powders, coversticks, rouge, lipsticks, lip contour sticks, makeup, nail varnish, glamor gel, etc.

“Dermocosmetics”, “cosmeceuticals” or “dermocosmetic compositions” or “dermocosmetic formulations” are compositions or formulations (i) for protecting against damage to skin or hair, (ii) for treating existing damage to skin or hair and (iii) for caring for skin or hair, comprising cosmetic skin, cosmetic nail, cosmetic hair, dermatological, hygiene or pharmaceutical compositions, preparations and formulations and decorative cosmetics. Also included are compositions for skin care in which the intended pharmaceutical dermatological use is achieved while taking into account cosmetic points of view. Compositions or formulations of this type are used for supporting, preventing and treating skin diseases and have a biological action in addition to the cosmetic effect. “Dermocosmetics” in the context of the above definition comprise, in a cosmetically tolerated medium, suitable assistants and additives which are familiar to the person skilled in the art and are described in handbooks of cosmetics, for example Schrader, Grundlagen und Rezepturen der Kosmetika, Hüthig Verlag, Heidelberg, 1989, ISBN 3-7785-1491-1, or Umbach, Kosmetik: Entwicklung, Herstellung und Anwendung kosmetischer Mittel, 2nd extended edition, 1995, Georg Thieme Verlag, ISBN 3 13 712602 9.

“Dermocosmetic active substances” in the context of the present invention are cosmetically tolerated substances, for example selected from the group consisting of the natural or synthetic polymers, pigments, humectants, oils, waxes, enzymes, minerals, vitamins, sunscreen agents, dyes, fragrances, antioxidants and preservatives and pharmaceutical active substances which are used for supporting, preventing and treating skin diseases and have a biological action which is curative, damage-preventing or regenerative or which improves the general condition of the skin.

“Deprotonated” in conjunction with the nitrogen atoms present in the piperidine ring of the compounds according to the invention means that these nitrogen atoms are present in tertiary form and are not present in a quaternary form as a result of the addition of a proton and do not carry a positive charge.

Cosmetic compositions for oral, dental and dental prosthesis care in the context of the present invention means all compositions or formulations and dosage forms suitable for oral, dental and dental prosthesis hygiene, as described in textbooks, e.g. Umbach: Kosmetik: Entwicklung, Herstellung und Anwendung kosmetischer Mittel, Chapter 7, pages 187-219, 2nd extended edition, 1995, Georg Thieme Verlag, ISBN 3 13 712602 9, which is hereby incorporated by reference. These compositions, formulations and dosage forms are familiar to the person skilled in the art and comprise, for example, tooth powders, tooth creams, toothpastes, children's tooth creams, tooth gels, liquid tooth creams, mouthwashes and mouthrinses, whereby this list should not be considered as definitive. The preparation of such compositions is familiar to the person skilled in the art and is generally described in textbooks (e.g. Umbach: Kosmetik: Entwicklung, Herstellung und Anwendung kosmetischer Mittel, 2nd extended edition, 1995, Georg Thieme Verlag, ISBN 3 13 712602 9). Thus, these compositions may also comprise further ingredients known to the person skilled in the art in addition to the compounds according to the invention according to the formulae 1-4. These may, for example, be surfactants, cleaning bodies, active substances, binders, humectants, consistency regulators, preservatives, dyes, flavors and sweeteners, whereby this list should not be considered as definitive. Said active substances may be active substances which prevent caries and gum inflammations. Fluoride may be mentioned in particular. Furthermore, these active substances can act, for example, against plaque bacteria and tartar formation, promote remineralization, desensitize sensitive tooth necks or protect the gum, whereby this list should not be considered as definitive. Reference is hereby made explicitly to the formulation examples shown on pages 205 to 207 in the textbook Umbach: Kosmetik: Entwicklung, Herstellung und Anwendung kosmetischer Mittel, 2nd extended edition, 1995, Georg Thieme Verlag, ISBN 3 13 712602 9.

“Cosmetically tolerated medium” should be broadly understood and means substances and mixtures thereof which are suitable for the preparation of cosmetic or dermocosmetic formulations.

“Cosmetically tolerated substances” do not lead to any irritations or damage oil contact with human or animal skin tissue or hair and have no incompatibilities with other substances. Furthermore, these substances have a low allergenic potential and are approved by national registration authorities for use in cosmetic formulations. These substances are familiar to the person skilled in the art and are described, for example, in handbooks of cosmetics, for example Schrader, Grundlagen und Rezepturen der Kosmetika, Hüthig Verlag, Heidelberg, 1989, ISBN 3-7785-1491-1.

“pH suitable for dermocosmetics” means a pH greater than 5, preferably from 5 to 8, also preferably 5.5-7.5, furthermore preferably 6-7.5, particularly preferably from 6.5 to 7.5, most preferably from 6.7 to 7.3. The suitable pH can change as a function of the application, the further constituents and the dosage form. The methods for determining the pH are familiar to the person skilled in the art and are described in appropriate textbooks, for example Schrader, Grundlagen und Rezepturen der Kosmetika, pages 944 to 945, Hüthig Verlag, Heidelberg, 1989, ISBN 3-7785-1491-1.

“Stabilization” in association with light- and/or oxidation-sensitive cosmetic ingredients or dermocosmetic active substances means an increased stability compared with compositions without compounds according to the invention according to the formulae 1 to 4, with otherwise identical composition and the same exposure to light or free radicals.

In association with oxidation-sensitive cosmetic ingredients or dermocosmetic active substances, “increased stability” means longer usability of the cosmetic or dermocosmetic compositions or prevention or reduction of spoiling or deactivation of the light- and/or oxidation-sensitive active substances as a result of, for example, light-induced oxidation damage, in comparison with compositions without compounds according to the invention according to the formulae 1 to 4, with otherwise identical composition and the same exposure to free radicals.

“Sterically hindered amines” in the context of the present invention are substances in which no hydrogen atoms are present in the α/α′ position relative to the nitrogen atom of the piperidine ring of the compounds according to the invention.

“Avoidance” in association with damage to the skin and hair caused by free radicals is (i) the prophylactic avoidance in the sense of prevention of said damage, (ii) a reduction in the actual damage caused by free radicals under acute exposure and (iii) the improved regeneration of the skin after damage produced by, for example, UV radiation. Avoidance is not necessarily to be understood as meaning 100% avoidance but may vary in its efficiency depending on the chosen formulations, the further ingredients, the concentrations present and the degree of exposure.

### DETAILED DESCRIPTION OF THE INVENTION

The present invention relates to dermocosmetics comprising at least one compound comprising the structural element of the general formula 1

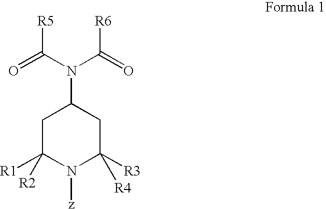

where

the radical Z has the following meaning: H, C₁-C₂₂-alkyl group, preferably C₁-C₁₂-alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl,

C₁-C₂₂-alkoxy group, preferably C₁-C₁₂-alkoxy group, such as alkoxymethyl, alkoxyethyl, alkoxypropyl, alkoxyisopropyl, alkoxybutyl, alkoxyisobutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyheptyl, alkoxyoctyl, alkoxynonyl, alkoxydecyl, alkoxyundecyl, alkoxydodecyl,

C₆-C₁₀-aryl group, such as phenyl and naphthyl, it being possible for the phenyl radical to be substituted by C₁- to C₄-alkyl radicals,

C₆- to C₁₀—O-aryl group which can be substituted by a C₁-C₂₂-alkyl or C₁-C₂₂-alkoxy group, preferably by a C₁-C₁₂-alkyl or C₁-C₁₂-alkoxy group as described above, and

the radicals R¹ to R⁶, independently of one another, have the following meaning: H, OH, O, C₁-C₂₂-alkyl group, preferably C₁-C₁₂-alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl,

C₁-C₂₂-alkoxy group, preferably C₁-C₁₂-alkoxy group, such as alkoxymethyl, alkoxyethyl, alkoxypropyl, alkoxyisopropyl, alkoxybutyl, alkoxyisobutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyheptyl, alkoxyoctyl, alkoxynonyl, alkoxydecyl, alkoxyundecyl, alkoxydodecyl,

C₆- to C₁₀-aryl group, such as phenyl and naphthyl, it being possible for the phenyl radical to be substituted by C₁- to C₄-alkyl radicals,

C₆- to C₁₀—O-aryl group which may be substituted by a C₁-C₂₂-alkyl or C₁-C₂₂-alkoxy group, preferably by a C₁-C₁₂-alkyl or C₁-C₁₂-alkoxy group as described above.

Particularly preferred alkyl radicals are methyl, ethyl, propyl, isopropyl, butyl, sec-butyl, tert-butyl, pentyl, isopentyl, neopentyl, tert-pentyl, hexyl, octyl, nonyl, dodecyl.

In the case of the alkoxy radicals, the radicals alkoxymethyl, alkoxyethyl, alkoxypropyl, alkoxyisopropyl, alkoxybutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyoctyl, alkoxynonyl, alkoxydodecyl are particularly preferred.

In a further embodiment, the radicals R⁵ and ⁶ of the compounds according to the invention are bridged so that a five- to eight-membered ring is formed, where this ring, if it is a five-membered ring, may be part of an oligomer as a result of covalent bonds at positions 3 and 4.

In a further preferred embodiment of the invention, the compound according to the general formula 1 is an HAS.

In a further preferred embodiment of the invention, the dermocosmetics comprise a compound of the general formula 2

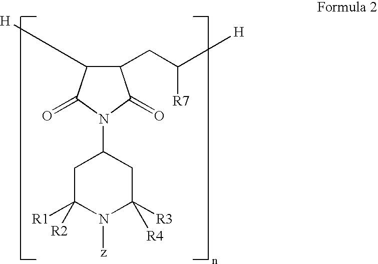

where the radicals Z and R¹ to R⁴, independently of one another, have the meaning stated in the description of the general formula 1 and

R⁷ corresponds to a C₁- to C₃₀-alkyl group, such as alkanes having an empirical formula C₄, C₂H₆, C₃H₈, C₄H₈, C₅H₁₀, C₆H₁₂, C₇H₁₄, C₈H₁₆, C₉H₁₈, C₁₀H₂₂, C₁₁H₂₄, C₁₂H₂₆, C₁₃H₂₈, C₁₄H₃₀, C₁₅H₃₂, C₁₆H₃₄, C₁₇H₃₆, C₁₈H₃₈, C₁₉H₄₀, C₂₀H₄₂, C₂₁H₄₄, C₂₂H₄₆, C₂₃H₄₈, C₂₄H₅₀, C₂₅H₅₂, C₂₆H₅₄, C₂₇H₅₆, C₂₈H₅₈, C₂₉H₆₀, C₃₀H₆₂, preferably straight-chain alkanes having the empirical formula C₁₅H₃₂, C₁₆H₃₄; C₁₇H₃₆, C₁₈H₃₈, C₁₉H₄₀, C₂₀H₄₂, C₂₁H₄₄, C₂₃H₄₈, C₂₄H₅₀, C₂₅H₅₂, particularly preferably C₁₈H₃₈, C₁₉H₄₀, C₂₀H₄₂, C₂₁H₄₄, C₂₂H₄₆, and n corresponds to an integer from 2 to 1000, 2 to 500, preferably 2 to 100, particularly preferably 2 to 50, very particularly preferably 2-25, more preferably 2-12, most generally preferably 2-10.

In a further preferred embodiment of the invention, the compound according to the general formula 2 is an HAS.

In a particularly preferred embodiment, the dermocosmetics comprise at least one sterically hindered amine of the formula 3

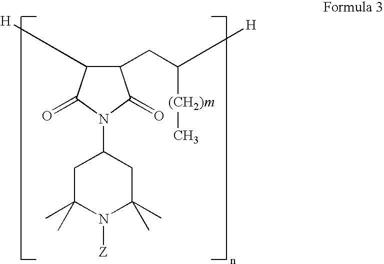

where Z corresponds to an H or a C₁-C₂₂-alkyl group, preferably C₁-C₁₂-alkyl group, such as alkanes having an empirical formula CH₄, C₂H₆, C₃H₈, C₄H₈, C₅H₁₀, C₆H₁₂, C₇H₁₄, C₈H₁₆, C₉H₁₈, C₁₀H₂₂, C₁₁H₂₄ or C₁₂H₂₆ and “n”corresponds to an integer from 2 to 1000, 2 to 500, preferably 2 to 100, particularly preferably 2 to 50, very particularly preferably 2-25, most preferably 2-12, most generally preferably 2-10, and “m” corresponds to an integer from 1 to 30, preferably the number 15, 16, 17, 18, 19, 20, 21, 22, 23, 24 or 25, particularly preferably from 18 to 21, more preferably from 19 to 21 or 20 or 21.

In a further preferred embodiment of the invention, the compound according to the general formula is a Uvinul®5050H.

The invention furthermore preferably relates to dermocosmetics comprising sterically hindered amines of the general formula 4

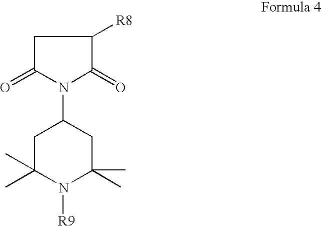

where R8 corresponds to a C₁-C₂₅-alkyl group, such as alkanes having an empirical formula CH₄, C₂H₆, C₃H₈, C₄H₈, C₅H₁₀, C₆H₁₂, C₇H₁₄, C₈H₁₆, C₉H₂₈, C₁₀H₂₂, C₁₁H₂₄, C₁₂H₂₆, C₁₃H₂₈, C₁₄H₃₀, C₁₅H₃₂, C₁₆H₃₄, C₁₇H₃₆, C₁₈H₃₈, C₁₉H₄₀, C₂₀H₄₂, C₂₁H₄₄, C₂₂H₄₆, C₂₃H₄₈, C₂₄H₅₀, C₂₅H₅₂, preferably C₅-C₁₅-alkyl group, such as alkanes having an empirical formula C₅H₁₀, C₆H₁₂, C₇H₁₄, C₈H₁₆, C₉H₁₈, C₁₀H₂₂, C₁₁H₂₄, C₁₂H₂₆, C₁₃H₁₃H₂₈, C₁₄H₃₀, C₁₅H₃₂, particularly preferably C₁₅-₂₅-alkyl group, such as alkanes having an empirical formula C₁₅H₃₂, C₁₆H₃₄, C₁₇H₃₆, C₁₈H₃₈, C₁₉H₄₀, C₂₀H₄₂, C₂₁H₄₄, C₂₂H₄₆, C₂₃H₄₈, C₂₄H₅₀, C₂₅H₅₂, and

R⁹ corresponds to an H, a C₁-C₂₂-alkyl group, preferably C₁-C₁₂-alkyl group, such as alkanes having an empirical formula CH₄, C₂H₆, C₃H₈, C₄H₈, C₅H₁₀, C₆H₁₂, C₇H₁₄, C₈H₁₆, C₉H₁₈, C₁₀H₂₂, C₁₁H₂₄, C₁₂H₂₆,

particularly preferably a C₁-C₈-alkyl group, such as alkanes having an empirical formula CH₄, C₂H₆, C₃H₈, C₄H₈, C₅H₁₀, C₆H₁₂, C₇H₁₄, C₈H₁₆, or

a C₁-C₂₂-alkoxy group, preferably C₁-C₁₂-alkoxy group, such as alkoxymethyl, alkoxyethyl, alkoxypropyl, alkoxyisopropyl, alkoxybutyl, alkoxyisobutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyheptyl, alkoxyoctyl, alkoxynonyl, alkoxydecyl, alkoxyundecyl, alkoxydodecyl, particularly preferably a C₁-C₈-alkoxy group, such as alkoxymethyl, alkoxyethyl, alkoxypropyl, alkoxyisopropyl, alkoxybutyl, alkoxyisobutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyheptyl, alkoxyoctyl.

The present invention particularly preferably relates to dermocosmetics comprising a sterically hindered amine selected from the group consisting of 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and 3-octenyl-N-(1,2,2,6,6-pentamethyl-4-piperidinyl)succinimide.

In another preferred embodiment the present invention also comprises cosmetic compositions for oral, dental and dental prosthesis care, these compositions comprising at least one compound according to the invention according to the formulae 1 to 4, preferably sterically hindered amines according to the formulae 1 to 4, particularly preferably 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide, 3-octenyl-N-(1,2,2,6,6-pentamethyl-4-piperidinyl)succinimide and/or Uvinul®5050H.

In a preferred embodiment of the present invention, the dermocosmetics or compositions for oral, dental and dental prosthesis care comprise an abovementioned compound according to the invention according to the formulae 1 to 4 in a concentration of from 0.001 to 1 percent by weight (% by weight), preferably from 0.0 1 to 0. 1% by weight, from 0.1 to 1% by weight, based on the total weight of the composition. In a further embodiment, the compositions comprise a compound according to the invention in a concentration of from 1 to 10% by weight, preferably from 2 to 8% by weight, from 3 to 7% by weight, from 4 to 6% by weight, based on the total weight of the composition. In a likewise preferred embodiment, the compositions comprise a compound according to the invention in a concentration of from 10 to 20% by weight, preferably from 11 to 19% by weight, 12 to 18% by weight, 13 to 17% by weight, 14 to 16% by weight, based on the total weight of the composition. In a further preferred embodiment, the compositions comprise a compound according to the invention in a concentration of from 20 to 30% by weight, preferably from 21 to 29% by weight, 22 to 28% by weight, 23 to 27% by weight 24 to 26% by weight, based on the total weight of the composition.

The invention furthermnore relates to dermocosmetics or compositions for oral, dental and dental prosthesis care comprising said compound according to the invention, the nitrogen atom of the piperidine ring of the compound being present in deprotonated form at the pH suitable for said compositions. In a preferred embodiment, the compounds according to the invention which are present in the dermocosmetics have a pKa in the range from 4 to 8, preferably from 6 to 7.5, particularly preferably from 6.5 to 7.3, most preferably from 6.8 to 7.2.

A particularly preferred embodiment of the present invention relates to dermocosmetics or compositions for oral, dental and dental prosthesis care comprising Uvinul®5050H (CAS No. 152261-33-1).

In another preferred embodiment, the dermocosmetics or compositions for oral, dental and dental prosthesis care, according to the invention, comprise, in addition to the abovementioned compounds according to the invention, one or more cosmetic or dermocosmetic active substances. These are preferably active substances selected from the group consisting of the natural or synthetic polymers, pigments, humectants, oils, waxes, enzymes, minerals, vitamins, sunscreen agents, dyes, fragrances, antioxidants, preservatives and/or pharmaceutical active substances which are used for supporting, preventing or treating skin diseases and have a biological action which is curative, damage-preventing or regenerative or which improves the general condition of the skin.

Suitable assistants and additives for preparing cosmetic hair or cosmetic skin formulations are familiar to the person skilled in the art and are described in handbooks of cosmetics, for example Schrader, Grundlagen und Rezepturen der Kosmetika, Hüthig Verlag, Heidelberg, 1989, ISBN 3-7785-1491-1, or Umbach, Kosmetik: Entwicklung, Herstellung und Anwendung kosmetischer Mittel, 2nd extended edition, 1995, Georg Thieme Verlag, ISBN 3 13 712602 9.

The dermocosmetics or compositions for oral, dental and dental prosthesis care according to the invention preferably comprise at least one of the compounds defined above and at least one constituent which differs therefrom and is selected from cosmetically active substances, emulsifiers, surfactants, preservatives, perfume oils, thickeners, hair polymers, hair and skin conditioners, graft polymers, water-soluble or dispersible silicone-containing polymers, light stabilizers, bleaches, gel formers, care compositions, colorants, tinting compositions, tanning compositions, dyes, pigments, consistency regulators, humectants, refatting agents, collagen, protein hydrolysis products, lipids, antioxidants, antifoams, antistatic agents, emollients and plasticizers. The active substances may also be present in encapsulated form in the cosmetic formulations, as described in the patents/patent applications EP 00974775 B1, DE 2311 712, EP 0278 878, DE 1999 47147, EP 0706822B1 and WO 98/16621 which are hereby incorporated by reference.

The antioxidants are advantageously selected from the group consisting of amino acids (e.g. glycine, histidine, tyrosine, tryptophan) and derivatives thereof, imidazoles (e.g. urocanic acid) and derivatives thereof peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, liponic acid and derivatives thereof (e.g. dihydroliponic acid), aurothioglucose, propylthiouracil and other thiols (e.g. thiorodoxin, glutathione, cysteine, cystine, cystamine and the glycosyl, N-acetyl, methyl, ethyl, propyl, amyl, butyl, and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximines, buthionine sulfones, penta, hexa and heptathionine sulfoximine), in very low tolerated doses (e.g. pmol to μmol/kg), and furthermore (metal)chelators (e.g. α-hydroxy-fatty acids, palmitic acid, phytic acid, lactoferrin), α-hydroxy acids (e.g. citric acid, lactic acid, malic acid), humic acid, gallic acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof vitamin C and derivatives thereof (e.g. sodium ascorbate, ascorbyl palmitate, magnesium ascorbyl phosphate, ascorbyl acetate), tocopherol and derivatives (e.g. vitamin E acetate, tocotrienol), vitamin A and derivatives (vitamin A palmitate) and coniferyl benzoate of benzoic resin, rutinic acid and derivatives thereof, α-glycosylrutin, ferulaic acid, furfurylideneglucitol, carnosine, butylhydroxytoluene, butylhydroxyanisole, nordihydroguajak resin acid, nordihydroguajaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenium methionine), stilbenes and derivatives thereof (e.g. stilbene oxide, trans-stilbene oxide).

The vitamins, provitamins or vitamin precursors of the vitamin B group which are preferably to be used according to the invention or derivatives thereof and the derivatives of 2-furanone include inter alia:

vitamin B₁, trivial name thiamine, chemical name 3-[(4′-amino-2′-methyl-5′-pyrimidinyl)methyl]-5-(2-hydroxyethyl)-4-methylthiazolium chloride.

Vitamin 2, trivial name riboflavin, chemical name 7,8-dimethyl-10-(1-D-ribityl)-benzo[g]pteridine-2,4(3H,10H)dione. In the free form, riboflavin occurs, for example, in whey, and other riboflavin derivatives can be isolated from bacteria and yeast. A riboflavin stercoisomer which is likewise suitable according to the invention is lyxoflavin which can be isolated from fish meal or liver and carries a D-arabityl radical instead of D-ribityl.

Vitamin B₃. This designation frequently means the compounds nicotinic acid and nicotinamide (niacinamide). Nicotinamide is preferred according to the invention.

Vitamin B₅ (pantothenic acid and panthenol). Panthenol is preferably used. Panthenol derivatives which can be used according to the invention are in particular the esters and ethers of panthenol and cationically derivatized panthenols. In a further preferred embodiment of the invention, derivatives of 2-furanone can also be used in addition to pantothenic acid or panthenol. Particularly preferred derivatives are the following substances which are also commercially available, dihydro-3 hydroxy-4,4-dimethyl-2(3H)furanone with the trivial name pantolactone (Merck), 4-hydroxymethyl-y-butyrolactone (Merck), 3,3-dimethyl-2-hydroxy-γ-butyrolactone (Aldrich) and 2,5-dihydro-5-methoxy-2-furanone (Merck), all stereoisomers expressly being included.

Advantageously, these compounds impart moisture-donating and skin-calming properties to the dermocosmetics according to the invention.

Vitamin B₆, which is to be understood as meaning not a single substance but 5 hydroxymethyl-2-methylpyridin-3-ol derivatives known by the trivial names pyridoxine, pyridoxamine and pyridoxal.

Vitamin B₇ (biotin), also designated as vitamin H or “skin vitamin”. Biotin is (3aS,4S, 6aR)-2-oxohexahydrothienol[3,4-d]imidazole-4-valeric acid.

Panthenol, pantolactone, nicotinamide and biotin are very particularly preferred according to the invention.

Dyes

Dyes which may be used are the substances suitable and approved for cosmetic purposes, as listed, for example, in the publication “Kosmetische Färbemittel” of the Dye Commission of the German Research Association, published by Verlag Chemie, Weinheim, 1984. These dyes are usually used in a concentration of from 0.001 to 0.1% by weight, based on the total mixture.

Pigments

In a preferred embodiment, the compositions according to the invention comprise at least one pigment.

The pigments are present in the product material in undissolved form and may be present in an amount of from 0.01 to 25% by weight particularly preferably from 5 to 15% by weight. The preferred particle size is from 1 to 200 μm, in particular from 3 to 150 μm, particularly preferably from 10 to 100 μm. The pigments are colorants which are virtually insoluble in the medium used and may be inorganic or organic. Mixed inorganic-organic pigments are also possible. Inorganic pigments are preferred. The advantage of the inorganic pigments is their excellent light stability, weather resistance and thermal stability. The inorganic pigments may be of natural origin, for example produced from chalk, ocher, umber, green earth, calcined Terra di Siena or graphite. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearl luster pigments and fluorescent or phosphorescent pigments, at least one pigment preferably being a colored, non-white pigment.

Metal oxides, metal hydroxides and hydrated metal oxides, mixed-phase pigments, sulfur-containing silicates, metal sulfides, complex metal cyanides, metal sulfates, chromates and molybdates and the metals themselves (bronze pigments) are suitable. Titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), hydrated chromium oxide (Cl 77289), iron blue (ferric ferrocyanides, CI7751 0), carmine (cochineal) are particularly suitable.

Mica-based pearl luster and colored pigments which are coated with a metal oxide or a metal oxychloride, such as titanium dioxide or bismuth oxychloride, and if appropriate further color-imparting substances, such as iron oxides, iron blue, ultramarine, carmine, etc, are particularly preferred, it being possible to determine the color by varying the coat thickness. Such pigments are sold, for example, under the trade names Rona®, Colorona®, Dichrona® and Timiron (Merck).

Organic pigments are, for example, the natural pigments sepia, gamboge, bone charcoal, Kassel brown, indigo, chlorophyll and other plant pigments. Synthetic organic pigments are, for example, azo pigments, anthraquinoids, indigoids and dioxazine, quinacridone, phthalocyanine, isoindolinone, perylene and perinone, metal complex, alkali blue and diketopyrrolopyrrole pigments.

In one embodiment, the composition according to the invention comprises from 0.01 to 10, particularly preferably from 0.05 to 5% by weight of at least one particulate substance. Suitable substances are, for example, substances which are solid at room temperature (25° C.) and are present in the form of particles. For example, silica, silicates, aluminates, aluminas, mica, salts, in particular inorganic metal salts, metal oxides, e.g. titanium dioxide, minerals and polymer particles are suitable.

The particles are present in the composition in undissolved, preferably stably dispersed form and after application to the surface used and evaporation of the solvent, can be deposited in solid form.

Preferred particulate substances are silica (silica gel, silicon dioxide) and metal salts, in particular inorganic metal salts, silica being particularly preferred. Metal salts are, for example, alkali metal or alkaline earth metal halides, such as sodium chloride or potassium chloride; alkali metal or alkaline earth metal sulfates, such as sodium sulfate or magnesium sulfate.

Pearl Luster Compositions

For example, the following are suitable as pearl luster compositions: alkylene glycol esters, especially ethylene glycol distearate; fatty acid alkanolamides, especially coconut fatty acid diethanolamide; partial glycerides, especially stearic acid monoglyceride; esters of polybasic, optionally hydroxy-substituted carboxylic acids with fatty alcohols having 6 to 22 carbon atoms, especially long-chain esters of tartaric acid; fatty substances, such as, for example, fatty alcohols, fatty ketones, fatty aldehydes, fatty ethers and fatty carbonates, where each have in total at least 24 carbon atoms, especially laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups and mixtures thereof

Conventional thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Nonionic thickeners are preferably used.

Suitable cosmetically and/or dermocosmetically active substances are, for example, color-imparting active substances, skin- and hair-pigmenting compositions, tinting compositions, tanning compositions, bleaches, keratin-hardening substances, antimicrobial active substances, light filter active substances, repellent active substances, substances having hyperemic activity, substances having keratolytic and keratoplastic activity, antidandruff active substances, antiphlogistic agents, substances having keratinizing activity, antioxidant active substances or substances active as free radical scavengers, skin-moisturizing substances or skin humectants, refatting active substances, substances having antierythematous or antiallergic activity, branched fatty acids, such as 18-methyleicosanoic acid, and mixtures thereof.

Artificially skin-tanning active substances which are suitable for tanning the skin without natural or artificial exposure to UV rays are, for example, dihydroxyacetone, alloxan and walnut shell extract. Suitable keratin-hardening substances are as a rule active substances as also used in antiperspirants, such as, for example, potassium aluminum sulfate, aluminum hydroxychloride, aluminum lactate, etc.

Antimicrobial active substances are used for destroying micoorganisms or inhibiting their growth and therefore serve both as a preservative and as a deodorant substance which reduces the formation or the intensity of body odor. These include, for example, conventional preservatives known to the person skilled in the art, such as p-hydroxybenzoic esters, imidazolidinylurea, formaldehyde, sorbic acid, benzoic acid, salicylic acid, etc. Such deodorant substances are, for example, zinc ricinoleate, triclosan, undecylenoic acid alkylolamides, triethyl citrate, chlorhexidine, etc.

The E numbers mentioned below are the designations customary in the directive 95/2/EEC.

The following may advantageously be used according to the invention as suitable preservatives.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | p-Hydroxybenzoic acid ethyl ester Na salt |
| E 216 | n-Propyl p-hydroxybenzoate |
| E 217 | p-Hydroxybenzoic acid n-propyl ester Na salt |
| E 218 | Methyl p-hydroxybenzoate |
| E 219 | p-Hydroxybenzoic acid methyl ester Na salt |
| E 220 | Sulfur dioxide |
| E 221 | Sodium sulfite |
| E 222 | Sodium hydrogen sulfite |
| E 223 | Sodium disulfite |
| E 224 | Potassium disulfite |
| E 226 | Calcium sulfite |
| E 227 | Calcium hydrogen sulfite |
| E 228 | Potassium hydrogen sulfite |
| E 230 | Biphenyl (diphenyl) |
| E 231 | Orthophenylphenol |
| E 232 | Sodium orthophenylphenolate |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

According to the invention preservatives or preservative assistants dibromodicyanobutane (2-bromo-2-bromomethylglutarodinitrile), 3-iodo-2-propynylbutyl carbamate, 2-bromo-2-nitropropane-1,3-diol, imidazolidinylurea, 5-chloro-2-methyl-4-isothiazolin-3-one, 2-chloroacetamide, benzalkonium chloride and benzyl alcohol customary in cosmetics are furthermore suitable.

Furthermore, phenyl hydroxyalkyl ethers, in particular the compound known under the name phenoxyethanol, are suitable as preservatives owing to their bactericidal and fungicidal effects on a number of microorganisms.

Other antimicrobial compositions are also suitable for being incorporated into the formulations according to the invention. Advantageous substances are, for example, 2,4,4′-trichloro-2′-hydroxydiphenyl ether (Irgasan), 1,6-di-(4-chlorophenylbiguanido)hexane (chlorhexidine), 3,4,4′-trichlorocarbanilide, quaternary ammonium compounds, clove oil, mint oil, thyme oil, triethyl citrate, farnesol (3,7,11-trimethyl-2,6,10-dodecatrien-1-ol) and the active substances and active substance combinations described in the laid-open patents DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 11, DE-196 31 003, DE-196 3 1004 and DE-196 34 019 and the patents DE-42 29 737, DE-42 37 081, DE-43 24 219, D)E-44 29 467, DE-44 23 410 and DE-195 16 705. Sodium bicarbonate may also advantageously be used. It is also possible to use microbial polypeptides.

Perfume Oils

The cosmetic compositions can, if appropriate, comprise perfume oils. Mixtures of natural and synthetic fragrances may be mentioned as perfume oils. Natural fragrances are extracts of blossoms (lily, lavender, rose, jasmine, neroli, ylang-ylang), stalks and leaves (geranium, patchouli, petitgrain), fruits (anise, coriander, caraway, juniper), fruit peels (bergamot, lemon, orange), roots (mace, angelica, celery, cardamom, costus, iris, calmus), woods (pinewood, sandalwood, guajak wood, cedar wood, rosewood), herbs and grasses (tarragon, lemongrass, sage, thyme), needles and branches (spruce, fir, pine, dwarf pine), resins and balsams (galbanum, elemi, benzoin, myrrh, olibanum, opoponax). Animal raw materials, such as, for example, civet and castoreum, are furthermore suitable. Typical synthetic fragrance compounds are products of the type consisting of the esters, ethers, aldehydes, ketones, alcohols and hydrocarbons. Fragrance compounds of the ester type are, for example, benzyl acetate, phenoxyethyl isobutyrate, 4-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonate, the ketones include, for example, the ionones, cc-isomethylionene and methyl cedryl ketone, the alcohols include anethol, citronellol, eugenol, isoeugenol, geraniol, linalool, phenylethyl alcohol and terioneol, the hydrocarbons include mainly the terpenes and balsams. However, mixtures of different fragrances which together produce an appealing fragrance note are preferably used. Essential oils of low volatility, which are generally used as aroma components, are also suitable as perfume oils, e.g. sage oil, chamomile oil, clove oil, balm oil, mint oil, cinnamon leaf oil, lime tree blossom oil, juniper oil, vetiver oil, oliban oil, galbanum oil, labolanum oil and lavandin oil. Bergamot oil, dihydromyrcenol, lilial, lyral, citronellol, phenylethyl alcohol, α-hexylcinnamaldehyde, geraniol, benzylacetone, cyclamenaldehyde, linalool, Boisambrene®Forte, ambroxan, indole, hedione, sandelice, lemon oil, mandarin oil, orange oil, allylamyl glycolate, cyclovertal, lavandin oil, muscatel sage oil, G39 damascone, Bourbon geranium oil, cyclohexyl salicylate, Vertofix®Coeur, iso-E-Super®, Fixolide®NP, evemyl, iraldein gamma, phenylacetic acid, geranyl acetate, benzyl acetate, rose oxide, romillate, irotyl and floramat, alone or as mixtures, are preferably used.

Oils, Fats and Waxes

The compositions according to the invention preferably comprise oils, fats and/or waxes, Constituents of the oil and/or fat phase of the compositions according to the invention are advantageously selected from the group consisting of the lecithins and of the fatty acid triglycerides, namely the triglyceryl esters of saturated and/or unsaturated, branched and/or straight-chain alkanecarboxylic acids having a chain length of 8 to 24, in particular 12 to 18, carbon atoms. The fatty acid triglycerides can advantageously be selected, for example, from the group consisting of the synthetic, semisynthetic and natural oils, such as, for example, olive oil, sunflower oil, soybean oil, peanut oil, rapeseed oil, almond oil, palm oil, coconut oil, castor oil, wheatgerm oil, grapeseed oil, safflower oil, evening primrose oil, macadamia nut oil and other similar ones. Further polar oil components can be selected from the group consisting of the esters of saturated and/or unsaturated, branched and/or straight-chain alkanecarboxylic acids having a chain length of 3 to 30 carbon atoms and saturated and/or unsaturated, branched and/or straight-chain alcohols having a chain length of 3 to 30 carbon atoms and from the group consisting of the esters of aromatic carboxylic acids and saturated and/or unsaturated, branched and/or straight-chain alcohols having a chain length of 3 to 30 carbon atoms. Such ester oils can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate dicaprylyl carbonate (Cetiol CC) and cocoglycerides (Myritol 331), butylene glycol dicaprylate/dicaprate and dibutyl adipate and synthetic, semisynthetic and natural mixtures of such esters, such as, for example, jojoba oil.

Furthermore, one or more oil components can advantageously be selected from the group consisting of the branched and straight-chain hydrocarbons and hydrocarbon waxes, of the silicone oils or of the dialkyl ethers, or from the group consisting of the saturated or unsaturated, branched or straight-chain alcohols.

Any desired mixtures of such oil and wax components can also advantageously be used in the context of the present invention. If appropriate, it may also be advantageous to use waxes, for example cetyl palmitate, as the sole lipid component of the oil phase.

According to the invention, the oil component is advantageously selected from the group consisting of 2-ethylhexyl isostearate, octyldodecanol, isotridecyl isononanoate, isoeicosane, 2-ethylhexyl cocoate, C12-15-alkyl benzoate, capryl/caproic acid triglyceride and dicaprylyl ether.

According to the invention, mixtures of C12-C15-allyl benzoate and 2-ethylhexyl isostearate, mixtures of C12-15-alkyl benzoate and isotridecyl isononanoate and mixtures of C12-15-alkyl benzoate, 2-ethylhexyl isostearate and isotridecyl isononanoate are advantageous.

Fatty acid triglycerides, in particular soybean oil and/or almond oil, are particularly preferably used according to the invention as oils having a polarity of from 5 to 50 mN/m.

Among the hydrocarbons, liquid paraffin, squalane and squalene can advantageously be used in the context of the present invention.

Furthermore, the oil phase can advantageously be selected from the group consisting of the Guerbet alcohols Guerbet alcohols are named after Marcel Guerbet, who described their preparation for the first time. They form according to the equation

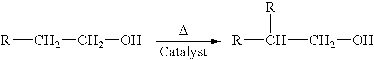

by oxidation of an alcohol to an aldehyde, by aldol condensation of the aldehyde, elimination of water from the aldol and hydrogenation of the allyl aldehyde. Guerbet alcohols are themselves liquid at low temperatures and cause virtually no skin irritations. They can advantageously be used as fatting, overfatting and also refatting constituents in cosmetic compositions.

The use of Guerbet alcohols in cosmetics is known per se. Such species are then generally distinguished by the structure

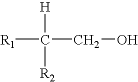

Here, R₁ and R₂ are as a rule straight-chain alkyl radicals. According to the invention, the Guerbet alcohol or alcohols is or are advantageously selected from the group where

R₁=propyl, butyl, pentyl, hexyl, heptyl or octyl and

R₂=hexyl, heptyl, octyl, nonyl, decyl, undecyl, dodecyl, tridecyl or tetradecyl.

Guerbet alcohols preferred according to the invention are 2-butyloctanol (for example commercially available as Isofol®12 (Condea)) and 2-hexyldecanol (for example, commercially available as Isofol®16 (Condea)).

Mixtures of Guerbet alcohols according to the invention can also advantageously be used according to the invention, such as, for example, mixtures of 2-butyloctanol and 2-hexyldecanol (for example, commercially available as Isofol®14 (Condea)).

Any desired mixture of such oil and wax components can also advantageously be used in the context of the present invention.

Among the polyolefins, polydecenes are the preferred substances.

The oil component can furthermore advantageously contain cyclic or linear silicone oils or consist completely of such oils, but, in addition to the silicone oil or the silicone oils, it is preferable to use an additional content of other oil phase components.

Low molecular weight silicones or silicone oils are as a rule defined by the following general formula:

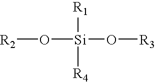

Higher molecular weight silicones or silicone oils are as a rule defined by the following general formula

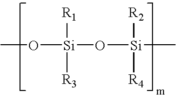

where the silicon atoms may be substituted by identical or different alkyl radicals and/or aryl radicals which are represented here generally by the radicals R₁ to R₄. The number of different radicals is, however, not necessarily limited to 4. m may assume values from 2 to 200 000.

Cyclic silicones advantageously to be used according to the invention are as a rule defined by the following general formula

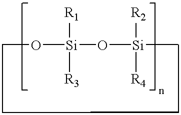

where the silicon atoms may be substituted by identical or different alkyl radicals and/or aryl radicals, which are represented here generally by the radicals R₁ to R₄. The number of different radicals is, however, not necessarily limited to 4. n may assume values from 3/2 to 20. Fractional values for n take account of the fact that uneven numbers of siloxyl groups may be present in the cycle.

Phenyltrimethicone is advantageously chosen as the silicone oil. Other silicone oils, for example dimethicone, hexamethylcyclotrisiloxane, phenyldimethicone, cyclomethicone (octamethylcyclotetrasiloxane), hexamethylcyclotrisiloxane, polydimethylsiloxane, poly(methylphenylsiloxane), cetyldimethicone and behenoxydimethicone, can also advantageously be used in the context of the present invention. Mixtures of cyclomethicone and isotridecyl isononanoate and those of cyclomethicone and 2-ethylhexyl isostearate are furthermore advantageous.

However, it is also advantageous to choose silicone oils having a constitution similar to that of the above-designated compounds, whose organic side chains are derivatized, for example polyethoxylated and/or polypropoxylated. These include, for example, polysiloxanepolyalkyl/polyether copolymers, such as, for example, cetyldimethicone copolyol.

Cyclomethicone (octamethylcyclotetrasiloxane) is advantageously used as the silicone oil to be used according to the invention.

Fat and/or wax components advantageously to be used according to the invention can be selected from the group consisting of the vegetable waxes, animal waxes, mineral waxes and petrochemical waxes. For example, candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes are advantageous.

Further advantageous fat and/or wax components are chemically modified waxes and synthetic waxes, such as, for example, Syncrowax®HRC (glyceryl tribehenate) and Syncrowax®AW 1 C (C₁₈₋₃₆ fatty acid) and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleate, such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as for example, hydrogenated soybean glyceride, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Certain organosilicon compounds which have physical properties similar to those of said fat and/or wax components, such as, for example, stearoxytrimethylsilane, are furthermore advantageous.

According to the invention, the fat and/or wax components can be used both individually and as a mixture in the compositions.

Any desired mixtures of such oil and wax components can also advantageously be used in the context of the present invention.

The oil phase is advantageously selected from the group consisting of 2-ethylhexyl isostearate, octyldodecanol, isotridecyl isononanoate, butylene glycol dicaprylate/dicaprate, 2-ethylhexyl cocoate, C₁₂₋₁₅-alkylbenzoate, caprylic/caproic acid triglyceride, dicaprylyl ether.

Mixtures of octyldodecanol, caprylic/caproic acid triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate and mixtures of C₁₂₋₁₅-alkyl benzoate, 2-ethylhexyl isostearate and isotridecyl isononanoate are particularly advantageous.

Among the hydrocarbons, liquid paraffin, cycloparaffin, squalane, squalene, hydrogenated polyisobutene and polydecene can advantageously be used in the context of the present invention.

The oil component is furthermore advantageously selected from the group consisting of the phospholipids. The phospholipids are phosphoric acid esters of acylated glycerols. Of the greatest importance among the phosphatidylcholines are, for example, the lecithins, which are distinguished by the general structure

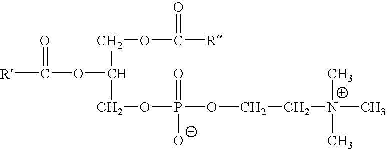

where R′ and R″ are typically straight-chain aliphatic radicals having 15 or 17 carbon atoms and up to 4 cis double bonds.

According to the invention, Merkur white oil, Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal) can be used as liquid paraffin which is advantageous according to the invention.

Suitable cosmetically tolerated oil and fat components are described in Karl-Heinz Schrader, Grundlagen und Rezepturen der Kosmetika, 2nd edition, Verlag Hüthig, Heidelberg, pages 319-355, which is hereby incorporated in its entirety by reference.

Solvents

If, in the context of the present invention, the cosmetic or dermatological composition is a solution or emulsion or dispersion the following may be used as solvents.

water or aqueous solutions, oils, such as triglycerides of caproic or of caprylic acid, but preferably castor oil; fats, waxes and other natural and synthetic fatty substances, preferably esters of fatty acids with alcohols having a small number of carbon atoms, for example with isopropanol, propylene glycol or glycerol, or esters of fatty alcohols with alkanoic acids having a small number of carbon atoms or with fatty acids;

alcohols, diols or polyols having a small number of carbon atoms, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

Surfactants

The compositions according to the invention may comprise surfactants. Examples of such surfactants are:
phosphoric acid esters and salts, such as, for example, DEA-oleth-10 phosphate and dilaureth-4 phosphate,
alkylsulfonates, for example sodium cocomonoglyceride sulfate, sodium C₁₂₋₁₄ olefin sulfonate, sodium laurylsulfoacetate and magnesium PEG-3 cocamide sulfate,
carboxylic acids and derivatives, such as, for example, lauric acid, aluminum stearate, magnesium alkanolate and zinc undecylenate, ester-carboxylic acids, for example calcium stearoyllactylate, laureth-6 citrate and sodium PEG-4 lauramide carboxylate,
esters which form by esterification of carboxylic acids with ethylene oxide, glycerol, sorbitan and other alcohols,
ethers, for example ethoxylated alcohols, ethoxylated lanolin, ethoxylated polysiloxanes, propoxylated POE ethers and alkylpolyglycosides, such as laurylglucoside, decylglycoside and cocoglycoside.

Polysorbates

Polysorbates may furthermore be incorporated into the composition.

Polysorbates advantageous in the context of the invention are
Polyoxyethylene(20) sorbitan monolaurate (Tween 20, CAS-No. 9005-64-5)
Polyoxyethylene(4) sorbitan monolaurate (Tween 21, CAS-No. 9005-64-5)
Polyoxyethylene(4) sorbitan monostearate (Tween 61, CAS-No. 9005-67-8)
Polyoxyethylene(20) sorbitan tristearate (Tween 65, CAS-No. 9005-71-4)
Polyoxyethylene(20) sorbitan monooleate (Tween 80, CAS-No. 9005-65-6)
Polyoxyethylene(5) sorbitan monooleate (Tween 81, CAS-No. 9005-65-5)
Polyoxyethylene(20) sorbitan trioleate (Tween 85, CAS-No. 9005-70-3).

Particularly advantageous are
Polyoxyethylene(20) sorbitan monopalmitate (Tween 40, CAS-No. 9005-66-7)
Polyoxyethylene(20) sorbitan monostearate (Tween 60, CAS-No. 9005-67-8).

These are advantageously used, according to the invention, in a concentration of from 0.1 to 5% by weight and in particular in a concentration of from 1.5 to 2.5% by weight, based on the total weight of the composition, individually or as a mixture of a plurality of polysorbates.

Conditioning Agents

In a preferred embodiment of the invention, the compositions also contain conditioning agents. Conditioning agents preferred according to the invention are, for example, all compounds which are mentioned in the International Cosmetic Ingredient Dictionary and Handbook (Volume 4, editors. R. C. Pepe, J. A. Wenninger, G. N. McEwen, The Cosmetic, Toiletry and Fragrance Association, 9th edition, 2002) under Section 4 under the keywords hair conditioning agents, humectants, skin-conditioning agents, skin-conditioning agents-emollient, skin-conditioning agents-humectant, skin-conditioning agents-miscellaneous, skin-conditioning agents-occlusive and skin protectants and all compounds mentioned in EP-A 934 956 (pages 11-13) under “water soluble conditioning agent” and “oil soluble conditioning agent”. Further advantageous conditioning agents are, for example, the compounds designated as polyquaternium according to INCI (in particular polyquaternium-1 to polyquaternium-56).

The suitable conditioning agents also include, for example, polymeric quaternary ammonium compounds, cationic cellulose derivatives and polysaccharides.

Conditioning agents advantageous according to the invention can be selected from the compounds shown in table 1 below.

TABLE 1
Conditioning agents advantageously to be used

| | | | Example |
|---|---|---|---|
| INCI designation | CAS number | Polymer type | (Tradename) |
| Polyquaternium-2 | CAS 63451-27-4 | Urea, N,N′-bis[3-(dimethylamino)propyl]polymer | Mirapol ® A-15 |
| | | with 1,1,′-oxybis(2-chloroethane) | |
| Polyquaternium-5 | CAS 26006-22-4 | Acrylamide, β-methacryloxyethyltriethylammonium | |
| | | methosulfate | |
| Polyquaternium-6 | CAS 26062-79-3 | N,N-Dimethyl-N-2-propenyl-2-propeneaminium | Merquat ® 100 |
| | | chloride | |
| Polyquaternium-7 | CAS 26590-05-6 | N,N-Dimethyl-N-2-propenyl-2-propeneaminium | Merquat ® S |
| | | chloride, 2-propeneamide | |
| Polyquaternium-10 | CAS 53568-66-4, | Quaternary ammonium salt of | Celquat ® SC-230M, |
| | 55353-19-0, | hydroxyethylcellulose | Polymer JR 400 |
| | 54351-50-7, | | |
| | 68610-92-4, | | |
| | 81859-24-7 | | |
| Polyquaternium-11 | CAS 53633-54-8 | Vinylpyrrolidone/dimethylaminoethyl methacrylate | Gafquat ® 755N |
| | | copolymer/diethyl sulfate reaction product | |
| Polyquaternium-16 | CAS 29297-55-0 | Vinylpyrrolidone/vinylimidazolinium | Luviquat ® HM552 |
| | | methochloride copolymer | |
| Polyquaternium-17 | CAS 90624-75-2 | | Mirapol ® AD-1 |
| Polyquaternium-19 | CAS 110736-85-1 | Qaternized water-soluble polyvinyl alcohol | |
| Polyquaternium-20 | CAS 110736-86-2 | Water-dispersible quaternized polyvinyl octadecyl | |
| | | ether | |
| Polyquaternium-21 | | Polysiloxane/polydimethyldimethylammonium | Abil ® B 9905 |
| | | acetate copolymer | |
| Polyquaternium-22 | CAS 53694-17-0 | Dimethyldiallylammonium chloride/acrylic acid | Merquat ® 280 |
| | | copolymer | |
| Polyquaternium-24 | CAS 107987-23-5 | Polymeric quaternary ammonium salt of | Quartisoft ® LM-20 |
| | | hydroxyethylcellulose | |
| Polyquaternium-28 | CAS 131954-48-8 | Vinylpyrrolidone/methacrylamidopropyltrimethylammonium | Gafquat ® HS-100 |
| | | chloride copolymer | |
| Polyquaternium-29 | CAS 92091-36-6, | Chitosan, which was reacted with propylene oxide | Lexquat ® CH |
| | 148880-30-2 | and quaternized with epichlorohydrin | |
| Polyquaternium-31 | CAS 136505-02-7, | Polymeric, quaternary ammonium salt, which is | Hypan ® QT 100 |
| | 139767-67-7 | prepared by reacting DMAPA acrylate/acrylic | |
| | | acid/acrylonitrogen copolymers and diethyl sulfate | |
| Polyquaternium-32 | CAS 35429-19-7 | N,N,N-Trimethyl-2-[(82-methyl-1-oxo-2- | |
| | | propenyl)oxy]ethaneaminium chloride polymer with | |
| | | 2-propeneamide | |
| Polyquaternium-37 | CAS 26161-33-1 | | |
| Polyquaternium-44 | | Copolymeric quaternary ammonium salt of | |
| | | vinylpyrrolidone and quaternized imidazoline | |

Further conditioners advantageous according to the invention are cellulose derivatives and quaternized guar gum derivatives, in particular guar hydroxypropylammonium chloride (e.g. Jaguar Excel®, Jaguar C 162® (Rhodia), CAS 65497-29-2, CAS 39421-75-5).

Nonionic poly-N-vinylpyrrolidone/polyvinyl acetate copolymers (e.g. Luviskol®VA 64 (BASF)), anionic acrylate copolymers (e.g. Luviflex@Soft (BASE)), and/or amphoteric amide-/acrylate/methacrylate copolymers (e.g. Amphomer® (National Starch)) can also advantageously be used according to the invention as conditioners.

Powder Raw Material

Addition of powder raw material may generally be advantageous. The use of talc is particularly preferred.

Ethoxylated Glyceryl Fatty Acid Esters

The dermocosmetics according to the invention or compositions for oral, dental and dental prosthesis care comprise, if appropriate, ethoxylated oils selected from the group consisting of the ethoxylated glyceryl fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil-PEG-7 ester, PEG-6 caprylic acid/caproic acid glycerides, PEG-10 olive oil glycerides, PEG-13 sunflower oil glycerides, PEG-7 hydrogenated castor oil, hydrogenated palm kernel oil glyceride-PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-35 castor oil, PEG-80 glyceryl cocoate, PEG-60 almond oil glycerides, PEG-60 “Evening Primrose” glycerides, PEG-200 hydrogenated glyceryl palmitate, PEG-90 glyceryl isostearate.

Preferred ethoxylated oils are PEG-7 glyceryl cocoate, PEG-9 coconut oil glycerides, PEG-40 hydrogenated castor oil, PEG-200 hydrogenated glyceryl palmitate.

Ethoxylated glyceryl fatty acid esters are used in aqueous cleaning formulations for different purposes. Glyceryl fatty acid esters having a low degree of ethoxylation (3-12 ethylene oxide units) are usually used as refatting agents for improving the skin sensation after drying out, while glyceryl fatty acid esters having a degree of ethoxylation of about 30-50 serve as solubilizers for nonpolar substances, such as perfume oils. Highly ethoxylated glyceryl fatty acid esters are used as thickeners. Common to all these substances is that they produce a particular skin sensation on the skin when used in dilution with water.

Light Protection Agents

In a particularly preferred embodiment of the present invention, these are sunscreen agents, in particular cosmetic or dermatological light protection agents which comprise at least one N filter substance.

The cosmetic and/or dermatological light protection compositions of this invention serve for cosmetic and/or dermatological light protection and furthermore for the treatment and care of the skin and/or the hair and as a makeup product in decorative cosmetics. They include, for example, sun creams, lotions, milks, oils, balsams and gels, lipcare products and lipsticks, cover creams and sticks, moisturizing creams, lotions and emulsions, face, body and hand creams, hair conditioners and rinses, hair setting compositions, styling gels, hair sprays, roll-on deodorants or eye wrinkle creams, tropicals, sunblockers and aftersun preparations. All preparations comprise at least one of said UV filter substances.

Sunscreen oils are generally mixtures of various oils with one or more light protection filters and perfume oils. The oil components are selected on the basis of different cosmetic properties. Oils which ensure a good fatting and impart a soft skin sensation, such as mineral oils (e.g. liquid paraffins) and fatty acid glycerides (e.g. peanut oil, sesame oil, avocado oil and medium-chain fatty triglycerides), are mixed with oils which improve the distributability and the absorption of the sunscreen oils into the skin, which reduce tack and make the oil film permeable to air and water vapor (perspiration). These include branched fatty acid esters (e.g. isopropyl palmitate) and silicon oils (e.g. dimetlhyl silicone). With the use of oils based on unsaturated fatty acids, antioxidants, e.g. E-tocopherol, are added in order to prevent said oils from becoming rancid. As a rule, sunscreen oils in the form of anhydrous formulations comprise no preservatives. Sunscreen milks and creams are prepared as oil-in-water (O/W) emulsions and as water-in-oil (W/O) emulsions. Depending on the type of emulsion the properties of the preparation are very different: O/W emulsions can be very easily distributed over the skin, are generally rapidly absorbed and can virtually always be easily washed off with water. W/O emulsions are more difficult to rub in, supply the skin with more fat and thus feel somewhat tackier but on the other hand protect the skin better from drying out. W/O emulsions are generally waterproof. In the case of O/W emulsions, the emulsion base, the choice of suitable light protection substances and, if appropriate, the use of assistants (e.g. polymers) decide the degree to which said emulsions are waterproof. In their composition, the bases of liquid and cream-like O/W emulsions resemble the other emulsions customary in skincare. Sunmilk should supply sufficient fat to the skin which has dried out owing to sun, water and wind. They must not be tacky, since this is felt to be particularly unpleasant in the heat and on contact with sand.

The light protection agents are as a rule based on a carrier which comprises at least one oil phase. However, compositions based on water alone are also possible. Accordingly, oils, oil-in-water and water-in-oil emulsions, creams and pastes, protective lipstick compositions and fat-free gels are suitable.

Suitable emulsions are, inter alia, also O/W macroemulsions, O/W microemulsions or O/W/O emulsions having surface-coated titanium dioxide particles present in dispersed form, the emulsions being obtainable by phase inversion technology, according to DE-A-197 26 121.

Conventional cosmetic assistants which may be suitable as additives are, for example (co)emulsifiers, fats and waxes, stabilizers, thickeners, biogenic active substances, film formers, fragrances, dyes, pearl luster compositions, preservatives, pigments, electrolytes (e.g. magnesium sulfate) and pH regulators.

Metal salts of fatty acids, such as, for example, magnesium, aluminum and/or zinc stearate, can be used as stabilizers.

Biogenic active substances are to be understood as meaning, for example, plant extracts, protein hydrolysis products and vitamin complexes.

Customary film formers are, for example, hydrocolloids, such as chitosan, microcrystalline chitosan and quaternized chitosan, polyvinylpyrrolidone, vinylpyrrolidone/vinyl acetate copolymers, polymers of the acrylic acid series, quaternary cellulose derivatives and similar compounds.

Suitable light filter active substances are substances which absorb UV rays in the UV-B and/or UV-A range. These are to be understood as meaning organic substances which are capable of absorbing ultraviolet rays and emitting the absorbed energy again in the form of longer-wave radiation, e.g. heat. The organic substances may be oil-soluble or water-soluble. Suitable UV filters are, for example, 2,4,6-triaryl-1,3,5-triazines in which the aryl groups may each carry at least one substituent which is preferably selected from hydroxyl, alkoxy, especially methoxy and alkoxycarbonyl, especially methoxycarbonyl and ethoxycarbonyl. p-Aminobenzoic acid esters, cinnamic acid esters, benzophenones, camphor derivatives and pigments which screen UV rays, such as titanium dioxide, talc and zinc oxide are furthermore suitable. Pigments based on titanium dioxide are particularly preferred.

For example, the following substances can be used as oil-soluble UV-B filters: 3-benzylidenecamphor and derivatives thereof, e.g. 3-(4-methylbenzylidene)camphor;

4-aminobenzoic acid derivatives, preferably 2-ethylhexyl 4-(dimethylamino)benzoate, 2-octyl 4-(dimethylamino)benzoate and amyl 4-(dimethylamino)benzoate;

ester of cinnamic acid, preferably 2-ethylhexyl 4-methoxycinnamate, propyl 4-methoxy-cinnamate, isoamyl 4-methoxycinnamate, isopentyl 4-methoxycinnamate and 2-ethylhexyl 2-cyano-3-phenylcinnamate (octocrylene);

esters of salicylic acid, preferably 2-ethylhexyl salicylate, 4-isopropylbenzyl salicylate, methyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di-2-ethylhexyl 4-methoxybenzalmalonate;

triazine derivatives, such as, for example, 2,4,6-trianilino(p-carbo-2′-ethyl-l′-hexyloxy)-1,3,5-triazine(octyltriazone) and dioctyl butamido triazone (Uvasorb® HEB):

propane-1,3-diones, such as, for example, 1-(4-tert-butylphenyl)-3-(4′-methoxyphenyl)propane-1,3-dione.

The following are suitable as water-soluble substances:

2-phenylbenzimidazole-5-sulfonic acid and the alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and the salts thereof.

The use of esters of cinnamic acid, preferably 2-ethylhexyl 4-methoxycinnamate, isopentyl 4-methoxycinnamate and 2-ethylhexyl 2-cyano-3-phenylcinnamate (octocrylene) is particularly preferred.

Furthermore, the use of derivatives of benzophenone, in particular 2-hydroxy-4-metboxybenzophenone, 2-hydroxy-4-methoxy-4′-methylbenzophenone and 2,2′-dihydroxy-4-methoxybenzophenone, and the use of propane-1,3-diones, such as, for example, 1-(4-tert-butylphenyl)-3-(4-′methoxyphenyl)propane-1,3-dione, is furthermore preferred.

The following are suitable as typical UV-A filters:

derivatives of benzoylmethane, such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenylpropane-1,3-dione;

aminohydroxyl-substituted derivatives of benzophenones, such as, for example, N,N-diethylaminobydroxybenzoyl-n-hexylbenzoate.

The UV-A and UV-B filters can of course also be used as mixtures.

Further suitable UV filter substances are mentioned in the following table.

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methylsulfate | 52793-97-2 |
| 3 | 3,3,5-Trimethylcyclohexyl salicylate | 118-56-9 |
| | (homosalatum) | |
| 4 | 2-Hydroxy-4-methoxybenzophenone | 131-57-7 |
| | (oxybenzonum) | |
| 5 | 2-Phenylbenzimidazole-5-sulfonic acid and its potassium, sodium and | 27503-81-7 |
| | triethanolamine salts | |
| 6 | 3,3′-(1,4-Phenylenedimethine)bis(7,7-dimethyl-2-oxobicyclo- | 90457-82-2 |
| | [2.2.1]heptane 1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzonum) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl-o-aminobenzoate or: | 134-09-8 |
| | 5-methyl-2-(1-methylethyl) 2-aminobenzoate | |
| 21 | Glyceryl p-aminobenzoate or: 1-glyceryl 4-aminobenzoate | 136-44-7 |
| 22 | 2,2′-Dihydroxy-4-methoxybenzophenone (dioxybenzone) | 131-53-3 |
| 23 | 2-Hydroxy-4-methoxy-4-methylbenzophenone (mexenone) | 1641-17-4 |
| 24 | Triethanolamine salicylate | 2174-16-5 |
| 25 | Dimethoxyphenylglyoxalic acid or: | 4732-70-1 |
| | 3,4-dimethoxyphenylglyoxalic acid sodium | |
| 26 | 3-(4′Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,-tetra- | 103597-45-1 |
| | methylbutyl)phenol] | |
| 30 | 2,2′-(1,4-Phenylene)bis-1H-benzimidazole-4,6-disulfonic acid, Na | 180898-37-7 |
| | salt | |
| 31 | 2,4-Bis[4-(2-ethylhexyloxy)-2-hydroxy]phenyl-6-(4-methoxy- | 187393-00-6 |
| | phenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | Disodium 2,2′-dihydroxy-4,4′-dimethoxybenzophenone- | 3121-60-6 |
| | 5,5′-disulfonate | |
| 36 | Benzoic acid, 2-[4-(diethylamino)-2-hydroxybenzoyl]hexyl ester | 302776-68-7 |
| 37 | 2-(2H-Benzotriazol-2-yl)-4-methyl-6-[2-methyl-3-[1,3,3,3-tetramethyl- | 155633-54-8 |
| | 1-[(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

In addition to the two abovementioned groups of primary light protection substances, it is also possible to use secondary light protection substances of the type consisting of the antioxidants, which break the photochemical reaction chain which is triggered if UV radiation penetrates the skin. Typical examples of these are superoxide dismutase, catalase, tocopherols (vitamin E) and ascorbic acid (vitamin C).

A further group comprises antiirritants which have an inflammation-inhibiting action on skin injured by UV light. Such substances are, for example, bisabolol, phytol and phytantriol.

The cosmetic and dermocosmetic formulations according to the invention can advantageously also comprise UV radiation-screen inorganic pigments based on metal oxides and/or other sparingly water-soluble or -insoluble metal compounds, selected from the group consisting of the oxides of zinc (ZnO), titanium (TiO₂), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides.

The inorganic pigments may be present in coated form, i.e. they have been treated on the surface. This surface treatment may, for example, consist of the pigments having been provided with a thin hydrophobic coat in a manner known per se, as described in DE-A-33 14 742.

Suitable repellent active substances are compounds which are capable of keeping away or of repelling certain animals, in particular insects. These include, for example, 2-ethyl-1,3-hexanediol, N,N-diethyl-m-toluamide, etc. Suitable substances having hyperemic activity which stimulate the blood flow of the skin are, for example, essential oils, such as mountain pine extract, lavender extract, rosemary extract, juniper berry extract, horse chestnut extract, birch leaf extract, hay blossom extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistic agents which counteract skin irritations are, for example, allantoin, bisabolol, dragostanol, chamomile extract, panthenol, etc.

The dermocosmetics according to the invention or compositions for oral, dental and dental prosthesis care may comprise, as a cosmetic and/or pharmaceutical active substance (as well as, if appropriate as an assistant), at least one cosmetically or pharmaceutically acceptable polymer which differs from the polymers which form the polyelectrolyte complex used according to the invention. These include very generally cationic, amphoteric and neutral polymers.

Suitable polymers are, for example, cationic polymers having the designation polyquaternium according to INCI, for example copolymers of vinylpyrrolidone/N-vinylimidazolium salts (Luviquat FC, Luviquat HM, Luviquat MS, Luviquat&commat, Care), copolymers of N-vinylpyrrolidone/dimethylaminoethyl methacrylate, quaternized with diethyl sulfate (Luviquat PQ 11), copolymers of N-vinylcaprolactam/N-vinylpyrrolidone/N-vinylimidazolium salts (Luviquat E. Hold), cationic cellulose derivatives (polyquaternium-4 and -10), acrylamido copolymers (polyquaternium-7) and chitosan.

Suitable cationic (quaternized) polymers are also Merquat (polymer based on dimethyldiallylammonium chloride), Gafquat (quaternized polymers which form by reaction of polyvinylpyrrolidone with quaternary ammonium compounds), polymer JR (hydroxyethylcellulose having cationic groups) and plant-based cationic polymers, e.g. guar polymers, such as the Jaguar brands from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyetlhyleneimines and salts thereof; polyvinylamines and salts thereof, cellulose derivatives, polyaspartic acid salts and derivatives. These include, for example, Luviflex 0 Swing (partly hydrolyzed copolymer of polyvinyl acetate and polyethylene glycol, from BASF).

Suitable polymers are also nonionic, water-soluble or water-dispersible polymers or oligomers, such as polyvinylcaprolactam, e.g. Luviskol 0 Plus (BASF), or polyvinylpyrrolidone and copolymers thereof, in particular with vinyl esters, such as vinyl acetate, e.g. Luviskol 0 VA 37 (BASF), polyamides, for example based on itaconic acid and aliphatic diamines, as described, for example, in DE-A-43 33 238.

Suitable polymers are also amphoteric or zwitterionic polymers, such as the octylarylamide/methyl methacrylate/tert-butylaminoethyl methacrylate-hydroxypropyl methacrylate copolymers obtainable under the names Amphomer (National Starch), and zwitterionic polymers as disclosed in the German patent applications DE 39 29 973, DE 21 50 557, DE 28 17 369 and DE 3708 451. Acrylamidopropyltrimethylammonium chloride/acrylic acid or methacrylic acid copolymers and the alkali metal and ammonium salts thereof are preferred zwitterionic polymers. Other suitable zwitterionic polymers are methacryloylethylbetaine/methacrylate copolymers which are commercially available under the name Amersette (AMERCHOL), and copolymers of hydroxyethyl methacrylate, methyl methacrylate, N,N-dimethylaminoethyl methacrylate and acrylic acid (Jordapon (D)).

Suitable polymers are also nonionic, siloxane-containing, water-soluble or water-dispersible polymers, e.g. polyethersiloxanes, such as Tegopren 0 (from Goldschmidt) or Besi&commat (from Wacker).

According to the invention, the dermocosmetic active substances (one or more compounds) can advantageously be selected from the group consisting of acetylsalicylic acid, atropine, azulene, hydrocortisone and derivatives thereof, e.g. hydrocortisone 17-valerate, vitamins of the B and D series, in particular vitamin B₁, vitamin B₁₂, vitamin D, vitamin A or derivatives thereof, such as retinyl palmitate, vitamin E or derivatives thereof, such as, for example, tocopheryl acetate, vitamin C and derivatives thereof, such as, for example, ascorbylglucoside, but also niacinamide, panthenol, bisabolol, polydocanol, unsaturated fatty acids, such as, for example, the essential fatty acids (usually referred to as vitamin F), in particular the γ-linolenic acid, oleic acid, eicosapentaenoic acid, docosahexaenoic acid and derivatives thereof, chloramphenicol, caffeine, prostaglandins, thymol, camphor, squalene, extracts of other products of vegetable and animal origin, e.g. evening primrose oil, borage oil or blackcurrant seed oil, fish oils, cod liver oil, but also ceramides and ceramide-like compounds, frankincense extract, green tea extract, water lilly extract, sweet wood extract, hamamelis, antidandruff substances (e.g. selenium disulfide, zinc pyrithione, piroctone, olamine, climbazole, octopirox, polydocanol and combinations thereof), complex active substances, such as, for example, those obtained from γ-oryzanol and calcium salts, such as calcium panthotenate, calcium chloride and calcium acetate.

It is also advantageous to select the active substances from the group consisting of the refatting substances, for example purcellin oil, Eucerit® and Neocerit®.

The active substance or substances is or are particularly advantageously furthermore selected from the group consisting of the NO synthase inhibitors, in particular if the formulations according to the invention are to be used for the treatment and prophylaxis of the symptoms of intrinsic and/or extrinsic skin aging and for the treatment and prophylaxis of the harmful effects of ultraviolet radiation on the skin and the hair. A preferred NO synthase inhibitor is nitroarginine.

The active substance or substances is or are furthermore advantageously selected from the group consisting of catechols and gallic acid esters of catechols and aqueous organic extracts of plants or plant parts which have a content of catechols or gallic acid esters of catechols such as, for example, the leaves of the plant family Theaceae, in particular of the species Camellia sinensis (green tea). The typical ingredients thereof (e.g. polyphenols or catechols, caffeine, vitamins, sugar, minerals, amino acids, lipids) are particularly advantageous.

Catechols constitute a group of compounds which are to be considered as hydrogenated flavones or anthocyanidinies and are derivatives of “catechol” (catechol, 3,3′,4′,5,7-flavanpentaol) 2-(3,4-dihydroxyphenyl)chroman-3,5,7-triol). Epicatechol ((2R,3R)-3,3′,4′,5,7-flavanpentaol) is an advantageous active substance in the context of the present invention.

Plant extracts having a content of catechols, in particular extracts of green tea, such as, for example, extracts of leaves of the plants of the species Camellia spec., very particularly of the tea varieties Camellia sinensis, C. assamica, C. taliensis and C. inawadiensis, and crosses of these with, for example, Camellia japonica are furthermore advantageous.

Preferred active substances are furthermore polyphenols or catechols from the group consisting of (−)-catechol, (+)-catechol, (−)-catechol gallate, (−)-gallocateclhol gallate, (+)-epicatechol, (−)-epicatechol, (−)epicatechol gallate, (−)-epigallo catechol, (−)-epigaIlocatechol gaIlate. Flavone and its derivatives (often also referred to collectively as “flavones”) are also advantageous active substances in the context of the present invention. They are characterized by the following basic structure (substitution positions indicated):

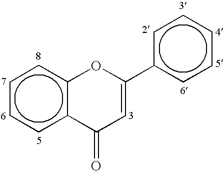

Some of the more important flavones which can also preferably be used in formulations according to the invention are shown in table 2 below.

### Table 2:

TABLE 2
Flavones

| | OH substitution position | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Campherol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones occur as a rule in glycosidated form. According to the invention, the flavonoids are preferably selected from the group consisting of the substances of the general formula

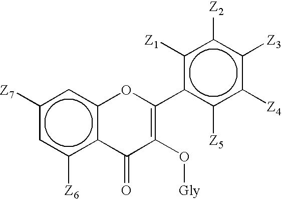

where Z₁ to Z₇, independently of one another, are selected from the group consisting of H, OH and alkoxy and hydroxyalkoxy groups, it being possible for the alkoxy and hydroxyalkoxy groups to be branched and straight-chain and to have 1 to 18 carbon atoms, and Gly being selected from the group consisting of the mono- and oligoglycoside radicals.

In addition, the active substances (one or more compounds) can also very advantageously be selected from the group consisting of the hydrophilic active substances, in particular from the following group:

α-hydroxy acids, such as lactic acid or salicylic acid, or the salts thereof, such as, for example, sodium lactate, calcium lactate, TEA lactate, urea, allantoin, serine, sorbitol, glycerol, milk proteins, panthenol, chitosan.

The amount of such active substances (one or more compounds) in the formulations according to the invention is preferably from 0.001 to 30% by weight, particularly preferably from 0.05 to 20% by weight, in particular from 1 to 10% by weight, based on the total weight of the formulation. Said and further active substances which can be used in the formulations according to the invention are stated in DE 103 18 526 A1 on pages 12 to 17, which is hereby incorporated by reference in its entirety.

The list of the stated ingredients which can be used in the formulations according to the invention is of course not to be considered as being definitive or limiting. The ingredients can be used individually or in any desired combinations with one another.

The compositions according to the invention are preferably skin protection compositions, skincare compositions, skin cleansing compositions, hair protection compositions, hair setting compositions, hair cleansing compositions, tooth powders, toothcreams, toothpastes, children's toothcreams, tooth gels, liquid toothcreams, mouthwashes and mouth rinses, or formulations for decorative cosmetics which are preferably used, depending on the field of use, in the form of ointments, creams, emulsions, suspensions, lotions, milk, pastes, gels, foams or sprays.

The formulation base of compositions according to the invention preferably comprises cosmetically or dermocosmetically/pharmaceutically acceptable assistants. The assistants which are known to be capable of being used in the area of pharmacy, of food technology and ancillary areas, in particular those listed in relevant pharmacopeias (e.g. DAB Ph. Fur. BP NF) and other assistants whose properties do not prevent physiological use are pharmaceutically acceptable.

Suitable assistants may be: lubricants, wetting agents, emulsifying and suspending compositions, preservatives, antioxidants, antiirritants, chelating agents, emulsion stabilizers, film formers, gel formers, odor masking compositions, resins, hydrocolloids, solvents, solubilizers, neutralizing agents, permeation accelerators, pigments, quaternary ammonium compounds, refatting and overfatting agents, ointment, cream or oil bases, silicone derivatives, stabilizers, sterilizing agents, blowing agents, drying agents, turbidity agents, thickeners, waxes, plasticizers and white oil. A development in this context is based on professional knowledge, as described, for example, in Fiedler, H. P. Lexikon der Hilfsstoffe für Pharmazie, Kosmetik und angrenzende Gebiete, 4th edition, Aulendorf: ECV-Editio-Kantor-Verlag, 1996.

For the preparation of the dermocosmetic compositions according to the invention, the active substances can be mixed or diluted with a suitable assistant (excipient). Excipients may be solid, semisolid or liquid materials which may serve as a vehicle, carrier or medium for the active substance. The admixing of further assistants is effected, if desired, in a manner known to the person skilled in the art. Furthermore, the polymers and dispersions are suitable as assistants in pharmacy, preferably as or in coating material(s) or binder(s) for solid dosage forms. They can also be used in creams and as tablet coating material and tablet binders.

Skin Cleansing Compositions

According to a preferred embodiment, the dermocosmetics according to the invention are skin cleansing compositions.

Preferred skin cleansing compositions are soaps of liquid or gel-like consistency, such as transparent soaps, luxury soaps, deodorant soaps, cream soaps, baby soaps, handcare soaps, abrasive soaps and syndets, pasty soaps, lubricating soaps and wash pastes, peeling soaps, moisturizing cloths, liquid wash, shower and bath preparations, such as wash lotions, shower baths and shower gels, foam baths, oil baths and scrubbing preparations, shaving foams, shaving lotions and shaving creams.

According to a further preferred embodiment, the compositions according to the invention are cosmetic compositions for the care and for the protection of the skin and hair, nailcare compositions and formulations for decorative cosmetics.

Suitable cosmetic skin compositions are, for example, facial toners, face masks, deodorants and other cosmetic lotions. Compositions for use in decorative cosmetics comprise, for example, coversticks, theater colors, mascara and eye shadows, lipsticks, kohl sticks, eyeliners, rouges, powders and eyebrow pencils.

In addition, the abovementioned compounds according to the invention, preferably sterically hindered amines according to the formulae 1 to 4, can be used in nose strips for pore cleansing, in antiacne compositions, repellents, shaving compositions, after- and pre-shave care compositions, aftersun care compositions, depilatory compositions, hair coloring compositions, intimate care compositions, and footcare compositions and in babycare.

The skincare compositions according to the invention are in particular W/O or O/W skin creams, day and night creams, eye creams, facial creams, antiwrinkle creams, sunscreen creams, humectant creams, bleaching creams, self-tanning creams, vitamin creams, skin lotions, care lotions and humectant lotions.

Cosmetic skin compositions and dermatological compositions preferably comprise at least one compound according to the invention according to the formulae 1 to 4, preferably sterically hindered amines according to the formulae 1 to 4, particularly preferably 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide, 3-octenyl-N(1,2,2,6,6-pentamethyl-4-piperidinyl)succinimide and/or Uvinul® 5050H in a proportion of from 0.001 to 1 percent by weight (% by weight), preferably from 0.01 to 0.1% by weight, from 0.1 to 1% by weight, based on the total weight of the composition. In a further embodiment, the compositions comprise the aboveinentioned compounds in a concentration of from 1 to 10% by weight, preferably from 2 to 8% by weight, from 3 to 7% by weight, from 4 to 6% by weight, based on the total weight of the composition. In a likewise preferred embodiment, the compositions comprise the abovementioned compounds in a concentration of from 10 to 20% by weight, preferably from 11 to 19% by weight, from 12 to 18% by weight, from 13 to 17% by weight, from 14 to 16% by weight, based on the total weight of the composition. In a further preferred embodiment, the compositions comprise the abovementioned compounds in a concentration of from 20 to 30% by weight, preferably from 21 to 29% by weight, from 22 to 28% by weight, from 23 to 27% by weight, from 24 to 26% by weight, based on the total weight of the composition.

In addition to the abovementioned compounds according to the invention and suitable carriers, the cosmetic skin formulations may comprise further active substances and assistants customary in skin cosmetics, as described above. These preferably include emulsifiers, preservatives, perfume oils, cosmetic active substances, such as phytantriol, vitamins A, E and C, retinol, bisabolol, panthenol, light protection agents, bleaches, colorants, tinting agents, tanning agents, collagen, protein hydrolysis products, stabilizers, pH regulators, dyes, salts, thickeners, gel formers, consistency regulators, silicones, humectants, refatting agents and/or further conventional additives.

Preferred oil and fatty components of the cosmetic skin compositions and dermocosmetic compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C6-C30 fatty acids, wax esters, such as, for example, jojoba oil, fatty alcohols, vaseline, hydrogenated lanolin and acetylated lanolin and mixtures thereof,

In order to establish certain properties, such as, for example, improvement of the sensation to the touch, of the spreading behavior, of the water resistance and/or of the binding of active substances and assistants, such as pigments, the cosmetic skin formulations and dermocosmetic formulations may additionally comprise conditioning substances based on silicone compounds.

Suitable silicone compounds are, for example, polyalkylsiloxanes, polyarylsiloxanes, polyarylalkylsiloxanes, polyethersiloxanes or silicone resins.

The preparation of the cosmetic or dermocosmetic formulations is effected by conventional processes known to the person skilled in the art.

The cosmetic and dermocosmetic compositions are preferably present in the form of emulsions, in particular as water-in-oil (W/O) or oil-ill-water (O/W) emulsions.

However, it is also possible to choose other types of formulations, for example gels, oils, oleogels, multiple emulsions, for example in the form of W/O/W or O/W/O emulsions, anhydrous ointments and ointment bases, etc. Emulsifier-free formulations, such as hydrodispersions, hydrogels and a Pickering emulsion, are also advantageous embodiments.

The preparation of emulsions is effected by known methods. In addition to at least one sterically hindered amine according to the invention, the emulsions comprise, as a rule, conventional constituents, such as fatty alcohols, fatty acid esters and in particular fatty acid triglycerides, fatty acids, lanolin and derivatives thereof, natural or synthetic oils or waxes and emulsifiers in the presence of water. The choice of the additives specific to the emulsion type and the preparation of suitable emulsions are described, for example, in Schrader, Grundlagen und Rezepturen der Kosmetika, Hüthig Buch Verlag, Heidelberg, 2nd edition, 1989, third part, or Umbach, Kosmetik: Entwicklung, Herstellung und Anwendung kosmetischer Mittel, 2nd extended edition, 1995, Georg Thieme Verlag, ISBN 3 13 712602 9, pages 122 et seq. which is hereby incorporated by reference.

A suitable emulsion as a W/O emulsion, for example for a skin cream, etc., generally comprises an aqueous phase which is emulsified by means of a suitable emulsifier system in an oil or fat phase. A polyelectrolyte complex can be used for providing the aqueous phase.

Preferred fat components which may be present in the fat phase of the emulsions are hydrocarbon oils, such as liquid paraffin, purcellin oil, perhydrosqualene and solutions of microcrystalline waxes in these oils, animal or vegetable oils, such as sweet almond oil, avocado oil, calophylum oil, lanolin and derivatives thereof, castor oil, sesame oil, olive oil, jojoba oil, karite oil, hoplostethus oil, mineral oils whose distillation under atmospheric pressure begins at about 250° C. and whose distillation end point is 410° C., such as, for example, vaseline oil, esters of saturated or unsaturated fatty acids, such as alkyl myristates, e.g. isopropyl, butyl or cetyl myristate, hexadecyl stearate, ethyl or isopropyl palmitate, octanoic or decanoic acid triglycerides and cetyl ricinoleate.

The fat phase may also comprise soluble silicone oils, such as dimethylpolysiloxane, methylphenylpolysiloxane and the silicone glycol copolymer, fatty acids and fatty alcohols, in other oils.

In addition to the above-described compounds according to the invention, the skin care compositions may also comprise waxes, such as, for example, carnauba wax, candililla wax, beeswax, microcrystalline wax, ozokerite wax and calcium, magnesium and aluminum oleates, myristates, linoleates and stearates.

Furthermore, an emulsion according to the invention may be present as an O/W emulsion. Such an emulsion usually comprises an oil phase, emulsifiers which stabilize the oil phase in the aqueous phase, and an aqueous phase which is usually present in thickened form. Preferred emulsifiers are O/W emulsifiers, such as polyglyceryl esters, sorbitan ester and partially hydrolyzed glycerides.

According to a further preferred embodiment, the compositions according to the invention are a light protection agent, a shower gel, a shampoo formulation or a bath preparation, light protection preparations being particularly preferred.

Such formulations comprise at least one compound of the general formula 1 to 4 according to the invention and usually anionic surfactants as base surfactants and amphoteric and/or nonionic surfactants as cosurfactants. Further suitable active substances and/or assistants are generally selected from lipids, perfume oils, dyes, organic acids, preservatives and antioxidants and thickeners/gel formers, skin conditioning agents and humectants.

These formulations preferably comprise from 2 to 50% by weight, preferably from 5 to 40% by weight, particularly preferably from 8 to 30% by weight, based on the total weight of the formulation, of surfactants.

All anionic, neutral, amphoteric or cationic surfactants usually used in body cleansing compositions can be used in the wash, shower and bath preparations.

Suitable anionic surfactants are, for example, alkyl sulfates, alkyl ether sulfates, alkylsulfonates, alkylarylsulfonates, alkylsuccinates, alkylsulfosuccinates, N-alkoylsarcosinates, acyltaurates, acylisethionates, alkylphosphates, alkyl ether phosphates, alkyl ether carboxylates, alpha-olefinsulfonates, in particular the alkali metal and alkaline earth metal salts, e.g. sodium, potassium, magnesium and calcium, and ammonium and triethanolamine salts. The alkyl ether sulfates, alkyl ether phosphates and alkyl ether carboxylates may have from 1 to 10 ethylene oxide or propylene oxide units, preferably from 1 to 3 ethylene oxide units, in the molecule.

The include, for example, sodium lauryl sulfate, ammonium taurytsulfate, sodium lauryl ether sulfate, ammonium lauryl ether sulfate, sodium laurylsarcosinate, sodium oleylsuccinate, ammonium laurylsulfosuccinate, sodium dodecylbenzenesulfonate and triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example cocodimethylsulfopropylbetaine, laurylbetaine, cocamidopropylbetaine or sodium cocamphopropionate may be used.

Examples of suitable non ionic surfactants are the reaction products of aliphatic alcohols or alkylphenols having 6 to 20 carbon atoms in the alkyl chain, which may be linear or branched, with ethylene oxide and/or propylene oxide. The amount of alkylene oxide is from about 6 to 60 moles per mole of alcohol. Furthermore, alkylamine oxides, mono- or dialkylalkanolamides, fatty acid esters of polyethylene glycols, ethoxylated fatty acid amides, alkylpolyglycosides and sorbitan ether ester are suitable.

The wash, shower and bath preparations may also comprise conventional cationic surfactants, such as, for example quaternary ammonium compounds, for example cetyltrimethylammonium chloride.

Furthermore, the shower gel/shampoo formulation may comprise thickeners, such as, for example, sodium chloride, PEG-55, propylene glycol oleate, PEG-120-methylglucose dioleate and others, and preservatives, further active substances and assistants and water.

Hair Treatment Compositions

According to a further preferred embodiment, the compositions according to the invention are hair treatment compositions.

Hair treatment compositions according to the invention preferably comprise at least one compound according to the invention according to the formulae 1 to 4 in an amount in the range from about 0.01 to 30% by weight, preferably from 0.5 to 20% by weight, based on the total weight of the composition.

The hair treatment compositions according to the invention are preferably present in the form of a foam setting composition, hair mousse, hair gel, shampoo, hair spray, hair foam, split end fluid, straightening composition for permanent waves, hair colors and bleaches or “hot oil treatment”. Depending on the field of use, the cosmetic hair formulations can be applied as (aerosol) spray, (aerosol) foam, gel, gel spray, cream, lotion or wax. Hair sprays comprise both aerosol sprays and pump sprays without propellant. Hair foams comprise both aerosol foams and pump foams without propellant. Hair sprays and hair foams preferably predominantly or exclusively comprise water-soluble or water-dispersible components. If the compounds used in the hair sprays and hair foams according to the invention are water-dispersible, they can be used in the form of aqueous microdispersions having particle diameters of, usually, from 1 to 350 nm, preferably from 1 to 250 nm. The solids contents of these preparations are usually in a range from about 0.5 to 20% by weight. These microdispersions require as a rule no emulsifiers or surfactants for their stabilization.

In a preferred embodiment, the cosmetic hair formulations according to the invention comprise a) from 0.01 to 30% by weight of at least one compound of the general fornula 1, b) ftom 20 to 99.95% by weight of water and/or alcohol, c) from 0 to 50% by weight of at least one propellant, d) from 0 to 5% by weight of at least one emulsifier, e) from 0 to 3% by weight of at least one thickener, and up to 25% by weight of further constituents.

Alcohol is to be understood as meaning all alcohols customary in cosmetics, e.g. ethanol, isopropanol and n-propanol.

Further constituents are to be understood as meaning the additives customary in cosmetics, for example propellants, antifoams, surface-active compounds, e.g. surfactants, emulsifiers, foam formers and solubilizers. The surface-active compounds used may be anionic, cationic, amphoteric or neutral. Further conventional constituents may furthermore be, for example, preservatives, perfume oils, turbidity agents, active substances, UV filters, care substances, such as panthenol, collagen, vitamins, protein hydrolysis products, alpha- and beta-hydroxycarboxylic acids, stabilizers, pH regulators, dyes, viscosity regulators, gel formers, salts, humectants, refatting agents, complexing agents and further conventional additives.

These furthermore include all styling and conditioning polymers which are known in cosmetics and can be used in combination with the sterically hindered amines according to the invention if very special properties are to be established.

Suitable conventional polymers for hair cosmetics are, for example, the abovementioned cationic, anionic, neutral, nonionic and amphoteric polymers, which are hereby incorporated by reference.

For establishing certain properties, the formulations may additionally comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds, such as amodimethicones (CTFA).

Propellants are the propellants usually used for hair sprays or aerosol foams. Mixtures of propane/butane, pentane, dimethyl ether, 1,1-difluoroethane (HFC-152 a), carbon dioxide, nitrogen or compressed air, are preferred.

The formulation preferred according to the invention for aerosol hair foams comprises a) from 0.01 to 30% by weight of at least one compound of the general formula 1 according to the invention, preferably sterically hindered amines according to the formulae 1 to 4, b) from 55 to 99.8% by weight of water and/or alcohol, c) from 5 to 20% by weight of a propellant, d) from 0.1 to 5% by weight of an emulsifier and e) from 0 to 10% by weight of further constituents.

Emulsifiers which may be used are all emulsifiers usually used in hair foams. Suitable emulsifiers may be nonionic, cationic or anionic or amphoteric.

Examples of nonionic emulsifiers (INCI nomenclature) are laureths, e.g. laureth-4; ceteths, e.g. cetheth-1, polyethylene glycol cetyl ether, ceteareths, e.g. cetheareth-25, polyglycol fatty acid glycerides, hydroxylated lecithin, lactyl esters of fatty acids, alkylpolyglycosides.

Examples of cationic emulsifiers are cetyldimethyl-2-hydroxyethylammonium dihydrogen 50 phosphate, cetyltrimonium chloride, cetyltrimonium bromide, cocotrimoniummethyl sulfate, quaternium-1 bis x (NCI).

Anionic emulsifiers can be selected, for example, from the group consisting of the alkylsulfates, alkyl ether sulfates, alkylsulfonates, alkylarylsulfonates, alkylsuccinates, alkylsulfosuccinates, N-alkoylsarcosinates, acyltaurates, acylisethionates, alkylphosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates may have from 1 to 10 ethylene oxide or propylene oxide units, preferably from I to 3 ethylene oxide units, in the molecule,

A formulation suitable according to the invention for styling gels may have, for example, the following composition: a) from 0.01 to 30% by weight of at least one compound of the general formula 1 according to the invention, preferably sterically hindered amines according to the formulae 1 to 4, b) from 80 to 99.85% by weight of water and/or alcohol, c) from 0 to 3% by weight, preferably from 0.05 to 2% by weight, of a gel former, d) from 0 to 20% by weight of further constituents.

Gel formers which can may used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xantban gum, caprylic/caproic triglycerides, sodium acrylate copolymers, polyquaternium-32 (and) paraffinum liquidum (INCI), sodium acrylate copolymers (and) paraffinum liquidum (and) PPG-1 trideceth-6, acrylamidopropyltrimonium chloride/acrylamide copolymers, steareth-10 allyl ether, acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

A formulation comprising the compounds of the formula I according to the invention, preferably sterically hindered amines can preferably be used in shampoo formulations. Preferred shampoo formulations comprise a) from 0.0I to 30% by weight of at least one compound of the general formula 1 according to the invention, preferably sterically hindered amines according to the formulae 1 to 4, b) from 25 to 94.95% by weight of water, c) from 5 to 50% by weight of surfactants, d) from 0 to 5% by weight of a further conditioner and e) from 0 to 10% by weight of further cosmetic constituents.

In the shampoo formulations all anionic, neutral, amphoteric or cationic surfactants usually used in shampoos may be used.

Suitable anionic surfactants are, for example, alkylsulfates, alkyl ether sulfates, alkylsulfonates, alkylarylsulfonates, alkylsuccinates, alkylsulfosuccinates, N-alkoylsarcosinates, acyltaurates, acylisethionates, alkylphosphates, alkyl ether phosphates, alkyl ether carboxylates, alpha-olefinsulfonates, in particular the alkali metal and alkaline earth metal salts, e.g. sodium, potassium, magnesium and calcium, and ammonium and triethanolamine salts. The alkyl ether sulfates, alkyl ether phosphates and alkyl ether carboxylates may have from 1 to 10 ethylene oxide or propylene oxide units, preferably from 1 to 3 ethylene oxide units, in the molecule.

For example, sodium laurylsulfate, ammonium laurylsulfate, sodium lauryl ether sulfate, ammonium lauryl ether sulfate, sodium lauroylsarcosinate, sodium oleylsuccinate, ammonium laurylsulfosuccinate, sodium dodecylbenzenesulfonate and triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or -propionates or alkylamphodiacetates or -dipropionates.

For example, cocodimethylsulfopropylbetaine, laurylbetaine, cocamidopropylbetaine or sodium cocamphopropionate may be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is from about 6 to 60 moles per mole of alcohol. Furthermore, alkylamine oxides, mono- or dialkylalkanolamides, fatty acid esters of polyethylene glycols, alkylpolyglycosides or sorbitan ether ester are suitable.

The shampoo formulations may also contain conventional cationic surfactants, such as, for example, quaternary ammonium compounds, for example cetyltrimethylammonium chloride.

In the shampoo formulations conventional conditioners may be used in combination with the compounds of the general formula 1 according to the invention, preferably sterically hindered amines according to the formulae 1 to 4, in order to achieve certain effects.

These include, for example, the abovementioned cationic polymer having the name polyquaternium according to INCI, in particular copolymers of vinylpyrrolidone/N-vinylimidazolium salts (Luviquat FC, Luviquat&commat, HM, Luviquat MS, Luviquat Care), copolymers of N-vinylpyrrolidone/dimethylaminoethyl methacrylate quaternized with diethyl sulfate (Luviquat D) PQ 11), copolymers of N-vinylcaprolactam/N-vinylpyrrolidone/N-vinylimidazolium salts (Luviquat D Hold), cationic cellulose derivatives (polyquaternium-4 and -10), acrylamidcopolymers (polyquaternium-7). It is furthermore possible to use protein hydrolysis products, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and amino-functional silicone compounds, such as amodimethicones (CTFA). Furthermore, cationic guar derivatives, such as guarhydroxypropyltrimonium chloride (INCI), may be used.

According to a further embodiment, this cosmetic hair formulation or cosmetic skin formulation is used for the care or the protection of the skin or hair and is present in the form of an emulsion, a dispersion, a suspension, an aqueous surfactant formulation, a milk, a lotion, a cream, a balsam, an ointment, a gel, granules, a powder, a stick preparation, such as, for example, a lipstick, a foam, an aerosol or a spray. Such formulations are suitable for topical preparations. Suitable emulsions are oil-in-water emulsions and water-in-oil emulsions or microemulsions.

As a rule, the cosmetic hair formulation or cosmetic skin formulation is used for application to the skin (topically) or hair. Topical formulations are to be understood as meaning those formulations which are suitable for applying the active substances in fine distribution and preferably in a form absorbable by the skin to the skin, for example aqueous and aqueous alcoholic solutions, sprays, foams, foam aerosols, ointments, aqueous gels, emulsions of the O/W or W/O type, microemulsions or cosmetic stick preparations are suitable for this purpose.

According to a preferred embodiment of the cosmetic composition according to the invention, the composition comprises a carrier. Water, a gas, a water-based liquid, an oil, a gel, an emulsion or microemulsion, a dispersion or a mixture thereof is preferable as a carrier. Said carriers are well tolerated by the skin. Aqueous gels, emulsions or microemulsions are particularly advantageous for topical formulations.

Emulsifiers which may be used are nonionogenic surfactants, zwitterionic surfactants, ampholytic surfactants or anionic emulsifiers. The emulsifiers may be present in the composition according to the invention in amounts of from 0.1 to 10, preferably from 1 to 5, % by weight, based on the composition.

For example, a surfactant from at least one of the following groups may be used as the nonionogenic surfactant:

adducts of from 2 to 30 mol of ethylene oxide and/or from 0 to 5 mol of propylene oxide with linear fatty alcohols having 8 to 22 carbon atoms, with fatty acids having 12 to 22 carbon atoms and with alkylphenols having 8 to 15 carbon atoms in the alkyl group;

C_{12/18} fatty acid mono- and diester of adducts of from 1 to 30 mol of ethylene oxide with glycerol;

glyceryl mono- and diesters and sorbitan mono- and diesters of saturated and unsaturated fatty acids having 6 to 22 carbon atoms and the ethylene oxide adducts thereof;

alkyl mono- and -oligoglycosides having 8 to 22 carbon atoms in the alkyl radical and the ethoxylated analogs thereof;

adducts of from 15 to 60 mol of ethylene oxide with castor oil and/or hydrogenated castor oil;

polyol esters and in particular polyglyceryl esters, such as, for example, polyglyceryl polyricinoleate, polyglyceryl poly-12-hydroxystearate and polyglyceryl dimerate; mixtures of compounds from a plurality of these classes of substances are also suitable;

adducts of from 2 to 15 mol of ethylene oxide with castor oil and/or hydrogenated castor oil;

partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (e.g. sorbitol), alkylglucosides (e.g. methylglucoside, butylglucoside, laurylglucoside) and polyglucosides (e.g. cellulose);

mono-, di- and trialkyl phosphate and mono-, di- and/or Tri-PEG-alkyl phosphates and salts thereof; wool wax alcohols;

polysiloxane-polyalkyl-polyether copolymers or corresponding derivatives;

mixed esters of pentaerythritol, fatty acids, citric acid and fatty alcohol according to German patent 1165574 and/or mixed esters of fatty acids having 6 to 22 carbon atoms, methylglucose and polyols, preferably glycerol or polyglycerol, and polyalkylene glycols.

Zwitterionic surfactants can furthermore be used as emulsifiers. Zwitterionic surfactants are defined as those surface-active compounds which carry at least one quaternary ammonium group and at least one carboxylate or one sulfonate group in the molecule. Particularly suitable zwitterionic surfactants are the so-called betaines, such as the N-alkyl-N,N-dimethylammonium glycinates, for example cocoalkylalkyldimethylammonium glycinate, N-acylamino-propyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group and cocoacylaminoethylhydroxyethyl carboxymethylglycinate. The fatty acid amide derivative known under the CTFA name cocamidopropyl betaine is particularly preferred.

Other suitable emulsifiers are ampholytic surfactants. Ampholytic surfactants are understood as meaning those surface-active compounds which, in addition to a C_{8,18}-alkyl or C_{8,18}-acyl group in the molecule, comprise at least one free amino group and at least one —COOH or —SO₃H group and are capable of forming internal salts. Examples of suitable ampholytic surfactants are N-aIkyIgIycines, N-alkyIpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylamino acetic acids having in each case about 8 to 18 carbon atoms in the alkyl group.

Particularly preferred ampholytic surfactants are N-cocoalkylamino propionate, cocoacylaminoethylamino propionate and C_{12/18}-acylsarcosine. In addition to the ampholytic emulsifiers, quaternary emulsifiers are also suitable, those of the esterquat type being particularly preferred, preferably methyl-quaternized di-fatty acid triethanolamine ester salts. Furthermore, alkyl ether sulfates, monoglyceride sulfates, fatty acids sulfates, sulfosuccinates and/or ethercarboxylic acids may be used as anionic emulsifiers.

Suitable oily substances are Guerbet alcohols based on fatty alcohols having 6 to 18, preferably 8 to 10, carbon atoms, esters of linear C₆-C₂₂ fatty acids with linear C6-C22 fatty alcohols, esters of branched C₆-C₁₃-carboxylic acid with linear C₆-C₂₂ fatty alcohols, esters of linear C₆-C₂₂ fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₀ fatty acids, liquid mono-/di-, triglyceride mixtures based on C₆-C₁₈ fatty acids, esters of C₆-C₂₂ fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂ fatty alcohol carbonates, Guerbet carbonates, esters of benzoic acid with linear and/or branched C₆-C₂₂-alcohols (e.g. FinsoIv® TN), dialkyl ethers, ring-opening products of epoxidized fatty acid esters with polyols, silicone oils and/or aliphatic or naphthenic hydrocarbons. Other oily substances which may be used are silicone compounds, for example dimetlhylpolysiloxanes, methylphenylpolysiloxanes, cyclic silicones and amino-, fatty acid-, alcohol-, polyether-, epoxy-, fluorine-, alkyl- and/or glycoside-modified silicone compounds which may be present in either liquid or resin form at room temperature. The oily substances may be present in the compositions according to the invention in amounts of from 1 to 90, preferably from 5 to 80, and in particular from 10 to 50% by weight, based on the composition.

The invention furthermore relates to the use of the compounds of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5050H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide as an additive for the preparation of the cosmetic or dermocosmetic formulations described above, preferably skin protection compositions, skin care compositions, skin cleansing compositions, hair protection compositions, hair care compositions or hair cleansing compositions or in formulations for decorative cosmetics.

In a particular embodiment of the present invention, the compounds of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5050H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide are used as an additive for the preparation of said cosmetic or dermocosmetic formulation in order to stabilize the light- and/or oxidation-sensitive substances present therein, for example the abovementioned ones (e.g. vitamins, antioxidants, carotenoids, flavonoids, pharmaceutical active substances and/or plant extracts).

The invention furthermore relates to the use of the compounds of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5650H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide in dermocosmetics or cosmetic compositions for oral, dental and dental prosthesis care, for stabilizing the abovementioned light- and/or oxidation-sensitive dermatologically/pharmaceutically active substances, preferably for preventing damage to the active substances which is caused by free radicals, with the result that the stability or duration of use of the cosmetic or dermocosmetic compositions is increased,

A further preferred embodiment of the present invention relates to the use of the compounds of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5050OH, 3-dodecyl-N-(2,2,6,6tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide in dermocosmetics or cosmetic compositions for oral, dental and dental prosthesis care for avoiding damage caused by free radicals. The use, according to the invention, of the abovementioned compounds in cosmetic formulations provides, inter alia, protection from damage which is caused directly or indirectly by processes due to UV radiation or reactive compounds, such as, for example, aging of the skin, loss of skin moisture, loss of elasticity of the skin, development of wrinkles or furrows or pigment defects and age spots or brittle, dull and inelastic hair.

A further field of use for the compounds of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5050H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide, is for therapeutic or prophylactic use in certain diseases of the skin. Particular fields of use in this context are:
tumor diseases (e.g. melanomas, adenomas, etc.)
autoimmune diseases (e.g. PEMPHIGUS VULGARIS, BULLOUS PEMPHIGOID, SYSTEMIC LUPUS ERYTHEMATOSIS etc.)
sunburn.

The present invention furthermore relates to the use of the abovementioned formulations for the prevention of undesired changes to the appearance of the skin, such as, for example, acne or greasy skin, keratoses, rosaceae, light-sensitive, inflammatory, erythematous, allergic or autoimmune reactions.

Furthermore, the compounds of the general formula 1 according to the invention, preferably sterically hindered amines according to the formulae 1 to 4, particularly preferably those sterically hindered amines in which the nitrogen atom of the piperidine ring is present ill deprotonated form in a medium having a neutral or slightly acidic pH, more preferably the compounds of the general formula 4, most preferably the substances Uvinul®5050H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide, in cosmetic hair formulations, provide protection from premature aging processes of human hair and can thus be used as active substances in the cosmetic field of brittle, dull and inelastic hair.

The invention also relates to cosmetic formulations suitable for the protection of the human epidermis or human hair, wherein said formulations comprise, in a cosmetically suitable carrier, at least one compound of the general formula 1 according to the invention, preferably sterically hindered amines according to the formulae 1 to 4, particularly preferably those sterically hindered amines in which the nitrogen atom of the piperidine ring is present in deprotonated form in a medium having a neutral or slightly acidic pH, more preferably the compounds of the general formula 4, most preferably the substances Uvinul®5050H, 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide and/or 3-octenyl-N-(1,2,2,6,6-pentamethyl-4-piperidinyl)succinimide in a preferred concentration. In a further embodiment, the cosmetic formulations for the protection of the human epidermis or human hair comprise, in addition to the cosmetically suitable carrier and at least one of the abovementioned compounds, at least one further active substance or ingredient from the abovementioned active substances or ingredients.

For use, the cosmetic formulations according to the invention are applied in the manner customary for cosmetics or dermocosmetics to the skin, hair, gums and teeth.

In a further preferred embodiment, sterically hindered amines according to the formulae 1 to 4, in which the nitrogen atom of the piperidine ring is present in deprotonated form at the pH suitable for cosmetic or dermocosmetic compositions are chosen for the abovementioned uses. In a further preferred embodiment, these sterically hindered amines have a pKa in the range from 4to 8, preferably from 6 to 7.5, particularly preferably from 6.5 to 7.3, most preferably from 6.8 to 7.2.

The dermocosmetics or cosmetic compositions according to the invention, for oral, dental and dental prosthesis care may have the composition described above and serve for the preventive treatment, the care and the cleansing of the skin, hair, teeth or gums or as a makeup product in cosmetics. For the use, according to the invention, of the abovementioned compounds, the latter are added to the cosmetic or dermocosmetic compositions in a concentration of from 0.001 to 1 percent by weight (% by weight), preferably from 0.01 to 0.1% by weight from 0.1 to 1% by weight, based on the total weight of the composition. In a further embodiment of the present invention, the compounds mentioned according to the invention can be used in a concentration of from 1 to 10% by weight, preferably from 2 to 8% by weight, from 3 to 7% by weight from 4 to 6% by weight, based on the total weight of the composition. in a likewise preferred embodiment, the compounds mentioned according to the invention can be used in a concentration of from 10 to 20% by weight, preferably from 11 to 19% by weight, from 12 to 18% by weight, from 13 to 17% by weight, from 14 to 16% by weight, based on the total weight of the composition. In an embodiment of the invention which is also preferred, the compounds mentioned according to the invention can be used in a concentration from 20 to 30% by weight, preferably from 21 to 29% by weight from 22 to 28% by weight, from 23 to 27% by weight, from 24 to 26% by weight, based on the total weight of the composition.

### EXPERIMENTAL EXAMPLES

The following examples are disclosed in order to illustrate preferred embodiments of the present inventions These examples are not to be considered as definitive or as limiting the subject matter of the invention.

The following abbreviations are used in the experimental description:

(2-amino-2-methyl-propanol) AMP, (degrees Celsius) C°, (ethylenediamintetraacetic acid) EDTA, (hindered amine stabilizer) MS, (1,1-difluoroethane) HFC 152, (International Nomenclature of Cosmetic Ingredients) INCI, (milliliter) ml, (minutes) min.,(oil/water) O/W, (polyethylene glycol) PEG-25, (paraaminobenzoic acid) PABA, (parts per million) ppm, (quantum satis) q.s, (vinylpyrrolidones) VP, (water/oil) W/O, (active substance) AS, (polyvinylpyrrolidones) PVP.

### Example 1

### Oxidation Test Squalene Test

3 ml of a 2% strength solution of the azo initiator V65 (from Wako) are introduced together with 2500 ppm of test substance (based on the squalene solution) into 12×100 ml Fiolax test tubes and thoroughly mixed using a laboratory shaker. The samples are then stored open in a through-circulation drying oven for 30 days at 40° C. At intervals of 7 days, the squalene content of the samples is determined by means of the Raman spectrum and chemometric evaluation. The datapoints are documented in an XY graph and the efficiency of the test substance is determined from the slope of the decomposition curves in comparison with a reference sample without test substance. The slope of the reference sample divided by the slope of the test substance sample gives the factor Q_Raman.

| Structure/tradename | Qraman | pKa |
|---|---|---|
| Without stabilizer | 1 | |
| Tocopherol | 1.4 | |
| Ascorbic acid | 1.2 | |
| Rutin | 1.2 | |
| N—H, N-Alkyl compounds | | |
| 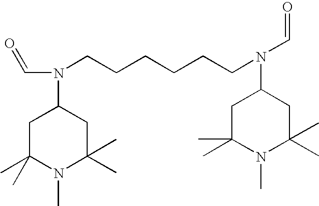 | 2.4 | |
| 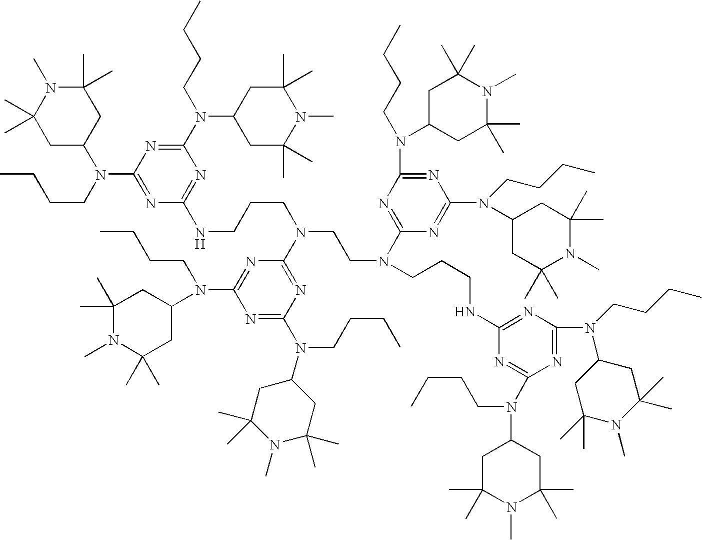 | 1.4 | |
| 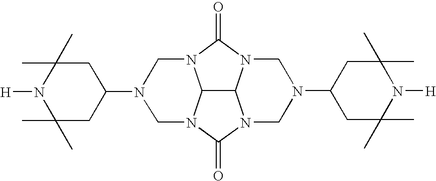 | 1.3 | 9.8²⁾ |
| 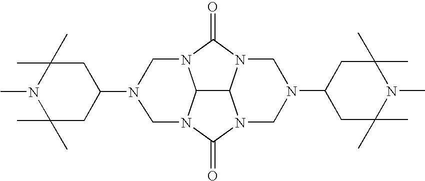 | 2.2 | |
| 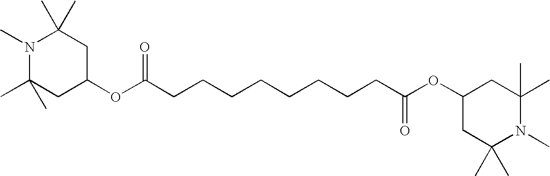 | 2.4 | 8.5¹⁾ |
| Tinuvin 292 | | |
| 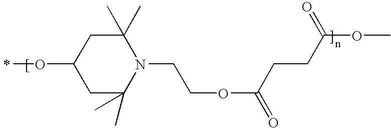 | 1.7 | 6.5⁴⁾ |
| idealized structural formula for Tinuvin 622 | | |
| N—O—R | | |
| 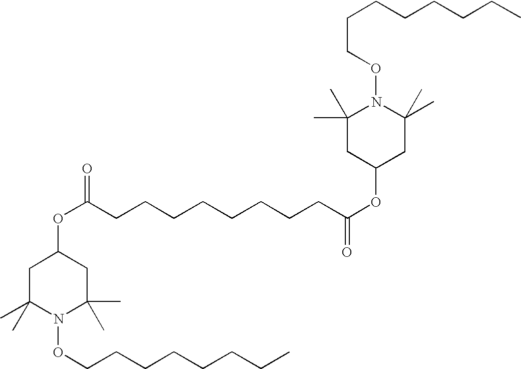 | 1.8 | 4.4¹⁾ |
| Tinuvin 123 | | |
| 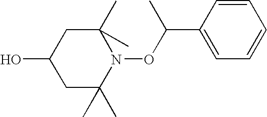 | 1.3 | |
| 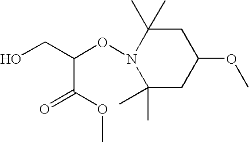 | 1.8 | |
| Compounds according to the invention | | |
| 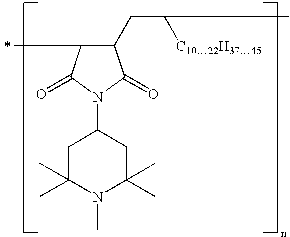 | 3.0 | |
| Compound 1 | | |
| for Idealized formula | 2.8 | 7.0³⁾ |
| 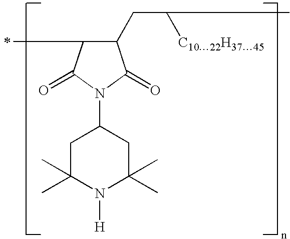 | | |
| Uvinul ® 5050H | | |
| 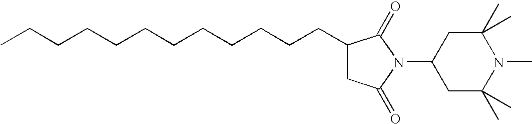 | 6.2 | |
| 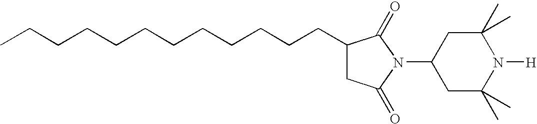 | 7.1 | |

Footnotes:
¹⁾Product selection guide for liquid industrial coatings, coil coatings and marine coatings, Ciba Specialty Chemicals, page 16 c-23/2005; stated as pKb value
²⁾Technical Information BASF AG; TI/P 3075 d of June 1999
³⁾Technical Information BASF AG; TI/P 3150 d of June 1999
⁴⁾H: Zweifel, in “Stabilization of Polymeric Materials-State-of-the-Art, Scope and Limitations”, page 15; Unesco School and dIUPAC Conference 2000

The results of this experiment show that the sterically hindered amines according to the invention have a stabilizing effect and can protect squalene from oxidation. Furthermore, it is found that the sterically hindered amines according to the invention have a substantially stronger effect for inhibiting oxidation damage compared with the comparative substances used. It is particularly remarkable that the sterically hindered amines according to the invention have an oxidation protection effect which is up to 5 times greater than that of the known oxidizing agents vitamins E and C.

### Example 2

### Solubility of Various HAS Compounds in Miglyol 812®

Experimental procedure: The respective MS compound was introduced gradually in small portions into 10 g of Miglyol and in each case stirred at room temperature until it had completely dissolved.

| | | | |
|---|---|---|---|
| 1) | Uvinul ® 5050 | → | min. 36% |
| 2) | Compound 1 (cf. table) | → | min. 33% |
| 3) | Uvinul ® 4050H | → | insoluble |
| 4) | Uvinul ® 4049 | → | insoluble |
| 5) | Tinuvin ® 770 | → | 4.7% |
| 6) | 2-Dodecyl-N-(2,2,6,6-tetramethyl-4- | → | min. 50% |
| | piperidinyl)succinimide | | |

The results of this experiment show that the sterically hindered amines according to the invention have good solubility in cosmetic oils. Furthermore, these experiments demonstrate that the comparative substances are virtually insoluble.

### Example 3

### Preparation of Uvinul® 5050

The preparation of the sterically hindered amine having the trade name Uvinul® 5050 is effected by a process disclosed in the example on page 6, lines 15 to 30, of the patent EP 0670 851 B1. The content of the patent EP 0670 851 B1 is hereby incorporated by reference in the present Application. The preparation of bis-(2,2,6,6-tetramethylpiperidyl)diimides is described in the patent EP 0031 3676 B1.

### Example 4

### Preparation of 3-octenyl-N-(2,2,6,6-tetramethyl-4-piperidinyl)succinimide

105.2 g of 3-octenylsuccinic anhydride were dissolved in 300 ml of glacial acetic acid at room temperature. 86 g of 2,2,6,6-tetramethyl-4-aminopiperidine were slowly added dropwise to this solution. The internal temperature increased to 70° C. during this procedure. Stirring was then carried out for a further 3 hours at 75-80° C. The glacial acetic acid was removed under reduced pressure and the residue was taken up in 500 ml of acetone, filtered off with suction and washed first with acetone and then water. 155.2 g of a colorless powder were obtained.

In the following examples, Uvinul 5050 H® (CAS No. 152261-33-1 produced and sold by BASF Aktiengesellschaft) and 3-dodecyl-N-(2,2,6,6-tetramethyl-4-piperidinyl)succinimide were used as compounds according the invention according to the formulae 1 to 4. Below, these two compounds are summarized by the designation “HAS according to the invention”.

### Example 5

### Use of the HAS According to the Invention in an Emulsion for Daycare—O/W type

| | |
|---|---|
| % | Ingredient (INCI) |

Active substance [AS] 1%: Uvinul 5050 H

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerol |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 1.0 | 1% HAS according to the invention |
| E | q.s. | Sodium Hydroxide |

AS 5%: Uvinul 5050 H

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerol |
| | 0.2 | Disodium EDTA |
| | 1.0 | Panthenol |
| | q.s. | Preservative |
| | 63.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, |
| | | Tocopherol, Retinol |
| | 5.0 | 5% HAS according to the invention |
| E | q.s. | Sodium Hydroxide |

Preparation: Heat the phases A and B separately from one another to about 80° C. Stir phase B into phase A and homogenize. Stir phase C into the combined phases A and B and once again homogenize. Cool to about 40° C. with stirring, add phase D, adjust the pH to about 6.5 with phase E, homogenize, and cool to room temperature with stirring.

Note. The formulations were prepared without inert gas. The filling must be effected into oxygen-impermeable packs, e.g. aluminum tubes.

### Example 6

### Use of the HAS According to the Invention in a protective Day Cream—O/W Type

| | |
|---|---|
| % | Ingredient (INCI) |

AS 1%: Uvinul 5050 H

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerol |
| | 0.2 | Disodium EDTA |
| | 1.0 | Panthenol |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | HAS according to the invention |
| E | q.s. | Sodium Hydroxide |

AS 5%: Uvinul 5050 H

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerol |
| | 0.2 | Disodium EDTA |
| | 1.0 | Panthenol |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | HAS according to the invention |
| E | q.s. | Sodium Hydroxide |

Preparation: Heat the phases A and B separately from one another to about 80° C. Stir phase B into phase A and homogenize. Incorporate phase C into the combined phases A and B and homogenize. Cool to about 40° C. with stirring. Add phase D, adjust the pH to about 6.5 with phase E and homogenize. Cool to room temperature with stirring.

### Example 7

### Use of the HAS According to the Invention in a Face Cleansing Lotion —O/W Type

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | HAS according to the invention |
| | 60.7 | Aqua dem. |

AS 5%: Uvinul 5050 H

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | HAS according to the invention |
| | 56.7 | Aqua dem. |

Preparation: Dissolve phase A. Stir phase B into phase A, incorporate phase C into the combined phases A and B. Dissolve phase D, stir into the combined phases A and B and C and homogenize. Stir for a further 15 min.

### Example 8

### Use of the HAS According to the Invention in a Daily Care Body Spray

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerol |
| | 1.0 | Tocopheryl Acetate |
| | 0.3 | Bisabolol |
| | 1.0 | HAS according to the invention |
| | 59.2 | Alcohol |

AS 5%: 3-Dodecyl-N-(2,2,6,6-tetramethyl-4-piperidinyl)succinimide

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerol |
| | 1.0 | Tocopheryl Acetate |
| | 0.3 | Bisabolol |
| | 5.0 | HAS according to the invention |
| | 55.2 | Alcohol |

Preparation: Weigh in the components of phase A and dissolve to give a clear solution.

### Example 9

### Use of the HAS According to the Invention in a Skin Care Gel

| | |
|---|---|
| % | Ingredient (INCI) |

AS 1%: Uvinul 5050 H

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | HAS according to the invention |
| | 75.4 | Aqua dem. |
| C | 0.8 | Triethanolamine |

AS 5%: 3-Dodecyl-N-(2,2,6,6-tetramethyl-4-piperidinyl)succinimide

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | HAS according to the invention |
| | 71.4 | Aqua dem. |
| C | 0.8 | Triethanolamine |

Preparation: Dissolve phase A to give a clear solution. Allow phase B to swell, and neutralize with phase C. Stir phase A into the homogenized phase B and homogenize.

### Example 10

### Use of the HAS According to the Invention in an After-Shave Lotion

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerol |
| | 1.0 | HAS according to the invention |
| | 0.1 | Triethanolamine |
| | 63.5 | Aqua dem. |

AS 5%: 3-Dodecyl-N-(2,2,6,6-tetramethyl-4-piperidinyl)succinimide

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerol |
| | 5.0 | HAS according to the invention |
| | 0.1 | Triethanolamine |
| | 59.5 | Aqua dem. |

Preparation: Mix the components of phase A. Dissolve phase B, incorporate into phase A and homogenize.

### Example 11

### Use of the HAS According to the Invention in an After-Sun Lotion

| | |
|---|---|
| % | Ingredient (INCI) |

AS 1%: Uvinul 5050 H

| | | |
|---|---|---|
| A | 0.4 | Acrylate/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerol |
| | 1.0 | HAS according to the invention |
| | 63.2 | Aqua dem. |
| C | 0.2 | Triethanolamine |

AS 5%: Uvinul 5050 H

| | | |
|---|---|---|
| A | 0.4 | Acrylate/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerol |
| | 5.0 | HAS according to the invention |
| | 59.2 | Aqua dem. |
| C | 0.2 | Triethanolamine |

Preparation: Mix the components of phase A. Stir phase B into phase A with homogenization. Neutralize with phase C and homogenize again.

### Example 12

### Use of the HAS According to the Invention in a Sunscreen Lotion

| | |
|---|---|
| % | Ingredient (INCI) |

AS 1%: Uvinul 5050 H

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerol |
| | 1.0 | Sodium Cetearyl Sulfate |
| | 0.5 | Xanthan Gum |
| | 59.7 | Aqua dem. |
| D | 1.0 | HAS according to the invention |
| | 1.0 | Phenoxyethanol, Methylparaben, Ethylparaben, Butylparaben, |
| | | Propyl-paraben, Isobutylparaben |
| | 0.3 | Bisabolol |

AS 5%: Uvinul 5050 H

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerol |
| | 1.0 | Sodium Cetearyl Sulfate |
| | 0.5 | Xanthan Gum |
| | 55.7 | Aqua dem. |
| D | 5.0 | HAS according to the invention |
| | 1.0 | Phenoxyethanol, Methylparaben, Ethylparaben, Butylparaben, |
| | | Propyl-paraben, Isobutylparaben |
| | 0.3 | Bisabolol |

Preparation: Heat the components of phases A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### Example 13

### Use of the HAS According to the Invention in a Sunscreen Lotion—O/W Type

| | |
|---|---|
| % | Ingredient (INCI) |

AS 1%: Uvinul 5050 H

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | HAS according to the invention |
| | 0.5 | Phenoxyethanol, Methylparaben, Butylparaben, Ethylparaben, |
| | | Propylparaben, Isopropylparaben |
| | 1.0 | Tocopheryl Acetate |
| | 0.2 | Bisabolol |

AS 5%: Uvinul 5050 H

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | HAS according to the invention |
| | 0.5 | Phenoxyethanol, Methylparaben, Butylparaben, Ethylparaben, |
| | | Propylpa raben, Isopropylparaben |
| | 1.0 | Tocopheryl Acetate |
| | 0.2 | Bisabolol |

Preparation: Heat phase A to about 80° C., stir in phase B and homogenize for 3 min. Also heat phase C to 80° C. and stir into the combined phases A and B with homogenization. Cool to about 40° C., stir in phase D and homogenize again.

### Example 14

### Use of the HAS According to the Invention in a Sunscreen Lotion—O/W Type

| | |
|---|---|
| % | Ingredient (INCI) |

AS 1%: Uvinul 5050 H

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerol |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | HAS according to the invention |
| | 1.0 | Tocopheryl Acetate |
| | 0.2 | Bisabolol |
| | q.s. | Perfume oil |
| | q.s. | Preservative |

AS 5%: Uvinul 5050 H

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerol |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | HAS according to the invention |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Also heat phase C to 80° C. and stir into the combined phases A and B with homogenization Cool to about 40° C., stir in phase D and homogenize again.

### Example 15

### Use of the HAS According to the Invention in a Foot Balsam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | HAS according to the invention |
| | 5.0 | Witch Hazel Extract |

AS 5%: Uvinul 5050 H

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | HAS according to the invention |
| | 5.0 | Witch Hazel Extract |

Preparation: Heat the components of phase A and B separately from one another to about 80° C. Stir phase B into phase A with homogenization. Cool to about 40° C. with stirring, add the phases C and D and homogenize again briefly. Cool to room temperature with stirring.

### Example 16

### Use of the HAS According to the Invention in a W/O Emulsion with Bisabolol

| | |
|---|---|
| % | Ingredient (INCI) |

AS 1%: Uvinul 5050 H

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerol |
| | 0.7 | Magnesium Sulfate |
| | 55.6 | Aqua dem. |
| C | 1.0 | HAS according to the invention |
| | 0.5 | Tocopheryl Acetate |
| | 0.6 | Bisabolol |

AS 5%: Uvinul 5050 H

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerol |
| | 0.7 | Magnesium Sulfate |
| | 51.6 | Aqua dem. |
| C | 5.0 | HAS according to the invention |
| | 0.5 | Tocopheryl Acetate |

Preparation: Beat the phases A and B separately from one another to about 85° C. Stir phase B into phase A and homogenize. Cool to about 40° C. with stirring. Add phase C and homogenize briefly again. Cool to room temperature with stirring.

### Example 17

### Foam Conditioner with Setting Composition

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 1.0 | HAS according to the invention |
| | 68.1 | Aqua dem. |
| | 10.0 | Propane/Butane |

AS 5%: Uvinul 5050 H

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 5.0 | HAS according to the invention |
| | 64.1 | Aqua dem. |
| | 10.0 | Propane/Butane |

Preparation: Weigh the components of phase A together, stir until everything has dissolved to give a clear-solution and fill.

### Example 18

### Foam Conditioner

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 1.0 | Polyquaternium-4 |
| | 0.5 | Hydroxyethyl Cetyldimonium Phosphate |
| | 1.0 | HAS according to the invention |
| | q.s. | Perfume oil |
| | q.s. | Preservative |
| | 91.5 | Aqua dem. |
| | 6.0 | Propane/Butane |

AS 5%: Uvinul 5050 H

| | | |
|---|---|---|
| A | 1.0 | Polyquaternium-4 |
| | 0.5 | Hydroxyethyl Cetyldimonium Phosphate |
| | 5.0 | HAS according to the invention |
| | q.s. | Perfume oil |
| | q.s. | Preservative |
| | 87.5 | Aqua dem. |
| | 6.0 | Propane/Butane |

Preparation: Weigh the components of phase A together, stir until everything has dissolved to give a clear solution and fill.

### Example 19

### Foam Conditioner

| | |
|---|---|
| % | Ingredient (INCI) |

AS 1%: Uvinul 5050 H

| | | |
|---|---|---|
| A | 1.0 | Polyquaternium-11 |
| | 0.5 | Hydroxyethyl Cetyldimonium Phosphate |
| | 1.0 | HAS according to the invention |
| | q.s. | Perfume oil |
| | q.s. | Preservative |
| | 91.5 | Aqua dem. |
| | 6.0 | Propane/Butane |

AS 5%: 3-Dodecyl-N-(2,2,6,6-tetramethyl-4-piperidinyl)succinimide

| | | |
|---|---|---|
| A | 1.0 | Polyquaternium-11 |
| | 0.5 | Hydroxyethyl Cetyldimonium Phosphate |
| | 5.0 | HAS according to the invention |
| | q.s. | Perfume oil |
| | q.s. | Preservative |
| | 87.5 | Aqua dem. |
| | 6.0 | Propane/Butane |

Preparation: Weigh the components of phase A together, stir until everything has dissolved to give a clear solution and fill.

### Example 20

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 0.5 | Laureth-4 |
| | q.s. | Perfume oil |
| B | 77.3 | Aqua dem. |
| | 10.0 | Polyquaternium-28 |
| | 1.0 | HAS according to the invention |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

AS 5%: Uvinul 5050 H

| | | |
|---|---|---|
| A | 0.5 | Laureth-4 |
| | q.s. | Perfume oil |
| B | 73.3 | Aqua dem. |
| | 10.0 | Polyquaternium-28 |
| | 5.0 | HAS according to the invention |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Make up with phase C.

### Example 21

### Styling Foam

| | |
|---|---|
| % | Ingredient (INCI) |

AS 1%: 3-Dodecyl-N-(2,2,6,6-tetramethy-4-piperidinyl)succinimide

| | | |
|---|---|---|
| A | 2.0 | Cocotrimonium Methosulfate |
| | q.s. | Perfume oil |
| B | 78.5 | Aqua dem. |
| | 6.7 | Acrylates Copolymer |
| | 0.6 | AMP |
| | 1.0 | HAS according to the invention |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

AS 5%: Uvinul 5050 H

| | | |
|---|---|---|
| A | 2.0 | Cocotrimonium Methosulfate |
| | q.s. | Perfume oil |
| B | 74.5 | Aqua dem. |
| | 6.7 | Acrylates Copolymer |
| | 0.6 | AMP |
| | 5.0 | HAS according to the invention |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Make up with phase C.

### Example 22

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.0 | Cocotrimonium Methosulfate |
| | q.s. | Perfume oil |
| B | 7.70 | Polyquaternium-44 |
| | 1.0 | HAS according to the invention |
| | q.s. | Preservative |
| | 79.3 | Aqua dem. |
| C | 10.0 | Propane/Butane |

AS 5%: Uvinul 505 H

| | | |
|---|---|---|
| A | 2.0 | Cocotrimonium Methosulfate |
| | q.s. | Perfume oil |
| B | 7.70 | Polyquaternium-44 |
| | 5.0 | HAS according to the invention |
| | q.s. | Preservative |
| | 75.3 | Aqua dem. |
| C | 10.0 | Propane/Butane |
| C. | | |

Preparation: Mix the components of phase A. Dissolve the components of phase B to give a clear solution and then stir phase B into phase A. Adjust the pH to 6-7 and make up with phase

### Example 23

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | HAS according to the invention |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

AS 5%: Uvinul 5050 H

| | | |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | HAS according to the invention |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation. Mix the components of phase A. Add the components of phase B one after the other and dissolve. Dissolve phase C in the mixture of A and B and then adjust the pH to 6-7. Make up with phase D.

### Example 24

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.00 | Cetrimonium Chloride |
| | q.s. | Perfume oil |
| B | 67.85 | Aqua dem. |
| | 7.00 | Polyquaternium-46 |
| | 1.00 | HAS according to the invention |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

AS 5%: Uvinul 5050 H

| | | |
|---|---|---|
| A | 2.00 | Cetrimonium Chloride |
| | q.s. | Perfume oil |
| B | 63.85 | Aqua dem. |
| | 7.00 | Polyquaternium-46 |
| | 5.00 | HAS according to the invention |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B and then adjust the pH to 6-7. Make up with phase D.

### Example 25

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | q.s. | PEG-40 Hydrogenated Castor Oil |
| | q.s. | Perfume oil |
| | 85.5 | Aqua dem. |
| B | 7.0 | Sodium Polystyrene Sulfonate |
| | 1.0 | HAS according to the invention |
| | 0.5 | Cetrimonium Bromide |
| | q.s. | Preservative |
| C | 6.0 | Propane/Butane |

Styling foam
AS 5%: Uvinul 5050 H

| | | |
|---|---|---|
| A | q.s. | PEG-40 Hydrogenated Castor Oil |
| | q.s. | Perfume oil |
| | 81.5 | Aqua dem. |
| B | 7.0 | Sodium Polystyrene Sulfonate |
| | 5.0 | HAS according to the invention |
| | 0.5 | Cetrimonium Bromide |
| | q.s. | Preservative |
| C | 6.0 | Propane/Butane |

Preparation: Solubilize phase A. Weigh phase B into phase A and dissolve to give a clear solution. Adjust the pH to 6-7 and make up with phase C.

### Example 26

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | q.s. | PEG-40 Hydrogenated Castor Oil |
| | q.s. | Perfume oil |
| | 92.0 | Aqua dem. |
| B | 0.5 | Polyquaternium-10 |
| | 1.0 | HAS according to the invention |
| | 0.5 | Cetrimonium Bromide |
| | q.s. | Preservative |
| C | 6.0 | Propane/Butane |

AS 5%: Uvinul 5050 H

| | | |
|---|---|---|
| A | q.s. | PEG-40 Hydrogenated Castor Oil |
| | q.s. | Perfume oil |
| | 88.0 | Aqua dem. |
| B | 0.5 | Polyquaternium-10 |
| | 5.0 | HAS according to the invention |
| | 0.5 | Cetrimonium Bromide |
| | q.s. | Preservative |
| C | 6.0 | Propane/Butane |

Preparation. Solubilize phase A. Weigh phase B into phase A and dissolve to give a clear solution. Adjust the pH to 6-7 and make up with phase C.

### Example 27

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | q.s. | PEG-40 Hydrogenated Castor Oil |
| | q.s. | Perfume oil |
| | 82.5 | Aqua dem. |
| B | 10.0 | Polyquaternium-16 |
| | 1.0 | HAS according to the invention |
| | 0.5 | Hydroxyethyl Cetyldimonium Phosphate |
| | q.s. | Preservative |
| C | 6.0 | Propane/Butane |

AS 5%: Uvinul 5050 H

| | | |
|---|---|---|
| A | q.s. | PEG-40 Hydrogenated Castor Oil |
| | q.s. | Perfume oil |
| | 78.5 | Aqua dem. |
| B | 10.0 | Polyquaternium-16 |
| | 5.0 | HAS according to the invention |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve to give a clear solution. Adjust the pH to 6-7 and make up with phase C.

### Example 28

### Styling Foam

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.0 | Cocotrimonium Methosulfate |
| | q.s. | Perfume oil |
| B | 84.0 | Aqua dem. |
| | 2.0 | Chitosan |
| | 1.0 | HAS according to the invention |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

AS 5%: Uvinul 5050 H

| | | |
|---|---|---|
| A | 2.0 | Cocotrimonium Methosulfate |
| | q.s. | Perfume oil |
| B | 80.0 | Aqua dem. |
| | 2.0 | Chitosan |
| | 5.0 | HAS according to the invention |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Preparation: Mix the components of phase A. Add the components of phase B one after the other and dissolve. Make up with phase C.

### Example 29

### Care Shampoo

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, Cocamide MEA, |
| | | Laureth-10 |
| | 1.0 | HAS according to the invention |
| | 7.7 | Polyquaternium-44 |
| | 2.0 | Amodimethicone |
| | q.s. | Perfume oil |
| | q.s. | Preservative |
| | 1.0 | Sodium Chloride |
| | 43.3 | Aqua dem. |
| B | q.s. | Citric Acid |

AS 5%: Uvinul 5050 H

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, Cocamide MEA, |
| | | Laureth-10 |
| | 5.0 | HAS according to the invention |
| | 7.7 | Polyquaternium-44 |
| | 2.0 | Amodimethicone |
| | q.s. | Perfume oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### Example 30

### Shower Gel

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 40.0 | Sodium Laureth Sulfate |
| | 5.0 | Decyl Glucoside |
| | 5.0 | Cocamidopropyl Betaine |
| | 1.0 | HAS according to the invention |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

AS 5%: Uvinul 5050 H

| | | |
|---|---|---|
| A | 40.0 | Sodium Laureth Sulfate |
| | 5.0 | Decyl Glucoside |
| | 5.0 | Cocamidopropyl Betaine |
| | 5.0 | HAS according to the invention |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation. Mix the components of phase A and dissolve. Adjust the pH to 6-7 with citric acid.

### Example 31

### Shampoo

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 44.6 | Aqua dem. |
| | 1.0 | HAS according to the invention |
| | 0.3 | Polyquaternium-10 |
| | 1.0 | Panthenol |
| | q.s. | Preservative |
| | 1.0 | Laureth-3 |
| | 2.0 | Sodium Chloride |

AS 5%: Uvinul 5050 H

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 40.6 | Aqua dem. |
| | 5.0 | HAS according to the invention |
| | 0.3 | Polyquaternium-10 |
| | 1.0 | Panthenol |
| | q.s. | Preservative |
| | 1.0 | Laureth-3 |
| | 2.0 | Sodium Chloride |

Preparation. Mix the components of phase A and dissolve. Adjust the pH to 6-7 with citric acid.

### Example 32

### Shampoo

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.00 | HAS according to the invention |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

AS 5%: Uvinul 5050 H

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 5.00 | HAS according to the invention |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7. Add phase B and heat to about 50° C. Cool to room temperature with stirring.

### Example 33

### Moisturizing Body Care Cream

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | HAS according to the invention |
| | 1.0 | Panthenol |
| | 0.5 | Magnesium Aluminum Silicate |
| | q.s | Preservative |
| | 65.5 | Aqua dem. |
| C | q.s. | Perfume oil |
| D | q.s. | Citric Acid |

AS 5%: Uvinul 5050 H

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 5.0 | HAS according to the invention |
| | 1.0 | Panthenol |
| | 0.5 | Magnesium Aluminum Silicate |
| | q.s | Preservative |
| | 61.5 | Aqua dem. |
| C | q.s. | Perfume oil |
| D | q.s. | Citric Acid |

Preparation: Heat the phases A and B separately to about 80° C. Homogenize phase B briefly, then stir phase B into phase A and homogenize again. Cool to about 40° C., add phase C and homogenize thoroughly again. Adjust the pH to 6-7 with citric acid.

### Example 34

### Moisturizing Body Care Cream

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | HAS according to the invention |

AS 5%: Uvinul 5050 H

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | HAS according to the invention |

Preparation: Heat the phases A and B separately to about 80° C. Stir phase B into phase A and homogenize. Cool to about 40° C. with stirring, add phase C and homogenize again. Allow to cool to room temperature with stirring.

### Example 35

### Liquid Makeup—O/W Type

| | |
|---|---|
| % | Ingredient (INCI) |

AS 1%: 3-Dodecyl-N-(2,2,6,6-tetramethyl-4-piperidinyl)succinimide

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | HAS according to the invention |
| | q.s. | Perfume oil |
| D | 5.7 | C.I. 77 891, Titanium Dioxide |
| | 1.1 | Iron Oxides |

AS 5%: Uvinul 5050 H

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | HAS according to the invention |
| | q.s. | Perfume oil |
| D | 5.7 | C.I. 77 891, Titanium Dioxide |
| | 1.1 | Iron Oxides |

Preparation: Heat the phases A and B separately to about 80° C. Stir phase B into phase A and homogenize. Cool to about 40° C. with stirring, add phases C and D and homogenize thoroughly again. Allow to coot to room temperature with stirring.

1-17. (canceled)

18. A dermocosmetic comprising at least one compound comprising a structural element of general formula (1) 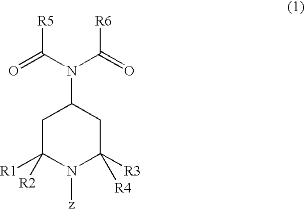
wherein
Z is H, C₁-C₂₂ alkyl, C₆-C₁₀ aryl, C₁-C₂₂ alkoxy, or C₆-C₁₀—O-aryl substituted with C₁-C₂₂alkyl or C₁-C₂₂ alkoxy; and
R¹ R² R³ R⁴ R⁵, and R⁶
are, independently of one another, H, C₁-C:₂₂ alkyl, C₆-C₁₀ aryl, O, OH, C₁-C₂₂ alkoxy, or C₆-C₁₀—O-aryl substituted with C₁-C₂₂ alkyl or C₁-C₁₂ alkoxy; and
wherein R⁵ and R⁶ are optionally bridged to define a five- to eight-membered ring, wherein when R⁵ and R⁶ are bridged to define a five-membered ring, said five-membered ring is optionally covalently bonded to an oligomer at positions 3 and 4.

19. The dermocosmetic of claim 18, wherein said structural element of general formula (1) is part of an oligomer having formula (2) 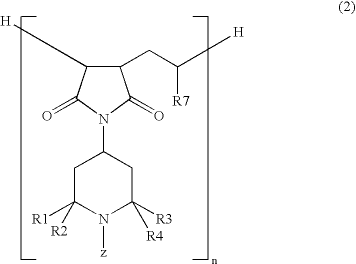
wherein
R⁷ is a C₁-C₃₀ alkyl; and
n is an integer from 2 to 1000.

20. The dermocosmetic of claim 18, wherein said structural element of general formula (1) is part of an oligomer having formula (3) 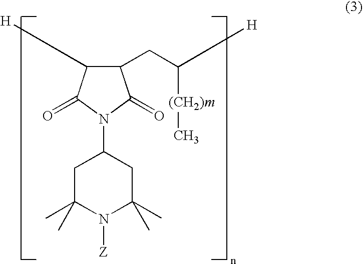
wherein
Z is H or C₁-C₂₂ alkyl group;
n is an integer from 2 to 1000; and
m is an integer from 1 to 30.

21. The dermocosmetic of claim 18, wherein said structural element of general formula (1) corresponds to part of a compound having formula (4) 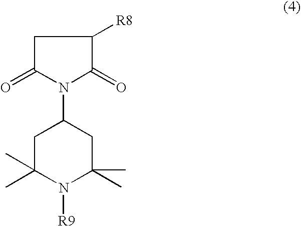
wherein
R⁸ is C₁-C₂₅ alkyl; and
R⁹ is H, C₁-C₂₂ alkyl, or C₁-C22 alkoxy.

22. The dermocosmetic of claim 18, wherein said at least one compound comprising a structural element of general formula (1) is selected from the group consisting of 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide, and 3-octenyl-N-(1,2,2,6,6-pentamethyl-4-piperidinyl)succinimide.

23. The dermocosmetic of claim 18, wherein said at least one compound comprising a structural element of general formula (1) is present in a concentration of from 0.001 to 30 % by weight, based on the total weight of the dermocosmetic formulation.

24. The dermocosmetic of claim 18, wherein the nitrogen atom of the piperidine ring is present in deprotonated form at the pH suitable for dermocosmetics.

25. The dermocosmetic of claim 18, wherein said at least one compound comprising a structural element of general formula (1) further comprises at least one further cosmetic and/or dermocosmetic active substance.

26. The dermocosmetic of claim 25, wherein said at least one further cosmetic and/or dermocosmetic active substance is selected from the group consisting of the natural or synthetic polymers, pigments, humectants, oils, waxes, enzymes, minerals, vitamins, sunscreen agents, dyes, fragrances, antioxidants, and preservatives.

27. The dermocosmetic of claim 18, wherein said dermocosmetic is in the form of an ointment, cream, emulsion, suspension, lotion, milk, paste, gel, foam, or spray.

28. The dermocosmetic of claim 27, wherein said dermocosmetic in the form of a skin protection composition, skin care composition, skin cleansing composition, hair protection composition, hair setting composition, hair cleansing composition, or formulation for decorative cosmetics.
